SEQUENCE LISTING (10 sequences)

SEQ ID NO: 1 - DNA, 21 bp, Photinus pyralis
cguacgcggaauacuucgatt

SEQ ID NO: 2 - DNA, 21 bp, Photinus pyralis
ucgaaguauuccgcguacgtt

SEQ ID NO: 3 - DNA, 21 bp, Photinus pyralis
cuuacgcugaguacuucgatt

SEQ ID NO: 4 - DNA, 21 bp, Photinus pyralis
ucgaaguacucagcguaagtt

SEQ ID NO: 5 - DNA, 21 bp, Aequorea victoria
gacguaaacggccacaagugc

SEQ ID NO: 6 - DNA, 21 bp, Aequorea victoria
cgcugcauuugccgguguuca

SEQ ID NO: 7 - protein, 33 aa, Bacteriophage T7
KNESSTNATNTKQWRDETKGFRDEARRFKNTAG

SEQ ID NO: 8 - protein, 26 aa, Apis florea
GIGAILKVLATGLPTLISWIKNKRKQ

SEQ ID NO: 9 - protein, 10 aa, Human immunodeficiency virus
GRKKRRQRRR

SEQ ID NO: 10 - protein, 16 aa, Drosophila melanogaster
RQIKIWFQNRRMKWKK

(19) United States
(12) Patent Application Publication
(10) Pub. No.: US 2004/0167090 A1
(43) Pub. Date: Aug. 26, 2004

(54) Covalent modification of RNA for in vitro and in vivo delivery

(76) Inventors: Sean D. Monahan, Madison, WI (US); Vladimir G. Budker, Middleton, WI (US); Lisa Nader, Madison, WI (US); Vladimir Subbotin, Madison, WI (US); Jon A. Wolff, Madison, WI (US)

Correspondence Address: Mark K. Johnson, Mirus Corporation, 505 S. Rosa Rd., Madison, WI, 53719, US

(21) Appl. No.: 10/782,075

(22) Filed: Feb. 19, 2004

Related U.S. Application Data
(60) Provisional application No. 60/448,789, filed on Feb. 21, 2003.
(60) Provisional application No. 60/455,724, filed on Mar. 18, 2003.

(51) Int. Cl. A61K 48/00; C12N 15/85

(52) U.S. Cl. 514/44; 435/455

(57) ABSTRACT

The post-synthetic modification of RNA for the delivery of the RNA to a mammalian cell is described. The modifications enhance resistant of the RNA to nuclease digestion and delivery of the RNA to the cell whether the RNA is delivered alone or in combination with a transfection agent. Activity of the RNA in the cell is maintained.

### CROSS-REFERENCE TO RELATED APPLICATIONS

This application claims the benefit of prior U.S. Provisional Application Serial No. 60/448,789 filed on Feb. 21, 2003, and 60/455,724 filed on Mar. 18, 2003.

### FIELD OF INVENTION

The present invention relates to methods and formulations for the delivery of oligonucleotides and small RNAs to cells in vitro and in vivo.

### BACKGROUND OF THE INVENTION

Recently, there has been a great deal of research interest in the delivery of RNA oligonucleotides to cells due to the discovery of RNA interference (RNAi). RNAi interference results in the knockdown of protein production within cells, via the interference of the small interfering RNA (siRNA) with the mRNA involved in protein production. This interference therefore curtails gene expression. The delivery of small double stranded RNAs (small interfering RNAs, or siRNAs, and microRNAs) to cells, has resulted in a greater than 80% knockdown of endogenous gene expression levels within the cell. Additionally, through the use of specific siRNAs, gene knockdown can be accomplished without inhibiting the expression of non-targeted genes.

A variety of methods have been employed for the delivery of the siRNA to cells including particle formation (complexation of the RNA with cationic polymers and lipids/liposomes) for in vivo and in vitro delivery, and naked RNA delivery in vivo. Currently, a variety of polycations have been tested for their ability to deliver siRNAs to cells. Although a great deal of effort is being directed toward complex formation and delivery, both efficient particle construction and the toxicity of the system remain problematic. For example, if one is trying to knockout an endogenous gene, any toxicity associated with the preparation or the delivery method can complicate the interpretation of the results.

Hamada and coworkers recently described the use of modified nucleotides in the sense strand, the antisense strand, and both strands, and the resulting influence on RNA interference. The modifications were incorporated into the RNA during synthesis of the RNA. Utilizing 2′-O,4′-C-ethylene thymidine (eT) and 2-hydroxyethylphosphate (HP) as the modifications, it was demonstrated that replacement of the 2 nucleotide 3′ overhangs with eT abolished RNAi, under all three substitution systems. However, following replacement of a 1 nucleotide 3′ overhang with HP on the sense strand, RNAi activity was retained. Similar modification to the antisense strand diminished RNAi activity (irrespective of sense strand modification).

Several additional modifications of the siRNA are known in the literature, for example, 2′-O-Alkyl modifications, 2′-halogen (especially fluorine), and a variety of amine base moieties on the ribose sugar. These examples require the synthesis of synthetic ribonucleosides for use in the RNA synthesis. This application details the post synthetic covalent modification of siRNAs for delivery to cells in vitro and in vivo.

### SUMMARY OF THE INVENTION

In a preferred embodiment, we describe chemical conjugates for inhibiting gene expression in a eukaryotic cells comprising, post-synthetically modified RNA oligonucleotides wherein the modifications are labile under mammalian physiological conditions. The modifications may be labile either through hydrolysis or enzymatic cleavage. We show that the modified oligonucleotides are effective in inducing RNAi and that the modifications enhance the delivery and/or effectiveness of the polynucleotide in inducing RNAi.

In a preferred embodiment, we describe processes for post-synthetic acylation of the 2′-OH group of an RNA backbone ribose to form a 2′-ester. The reaction can be conducted in aqueous or organic solvents. The modified RNA can be concentrated to dryness and redissolved in aqueous or organic solution. The acylating agent can be derived from an alkyl carboxylic acid (acid chloride, activated ester, etc.), or an anhydride or cyclic anhydride. Additionally, the acylating agent can possess a functional group selected from the list consisting of: hydrophobic groups, membrane active compounds, cell penetrating compounds, cell targeting signals, interaction modifiers, and steric stabilizers. Modification of an siRNA does not destroy the gene expression knockdown activity of the siRNA.

In a preferred embodiment, we describe post-synthetic modification of RNA comprising, reacting an RNA with a silyl chloride in an organic solvent. This reaction results in the formation of a modified RNA with the 2′-OH silylated to a silyl ether. Additional atoms on the RNA that may be modified by the silyl chloride include phosphate oxygens and nitrogen atoms on the nucleotide base. The silyl chloride can be an alkyl chlorosilane or a bischlorosilane. Additionally, the silylating agent can possess a functional group selected from the list consisting of: hydrophobic groups, membrane active compounds, cell penetrating compounds, cell targeting signals, interaction modifiers, and steric stabilizers. The modified RNA can be concentrated to dryness and redissolved in an aqueous or organic solution. Modification of the an siRNA with the silyl chloride does not destroy the gene expression knockdown activity of the siRNA.

In a preferred embodiment, we describe post-synthetic modification of RNA comprising, reacting the RNA with a alkylating agent selecting from the group consisting of nitrogen mustards, sulfur mustards, and activated three-membered ring containing molecules. These agents are known to react with nucleotide bases at the N7 atom of guanine and the N3 atom of adenine. The mustard or activated three-membered ring containing molecule can possess a functional group selected from the list consisting of: hydrophobic groups, membrane active compounds, cell penetrating compounds, cell targeting signals, interaction modifiers, and steric stabilizers. Activated three-membered rings containing molecules can be selected from the list consisting of: epoxides, cyclopropanes, and episulfides which possess a pendent group including but not limited to an amine, alkyl group, peptide, carboxylic acid, aldehyde, and ketone. The modified RNA obtained from the alkylation reaction can be taken up in aqueous or organic solution. Modification of an siRNA does not destroy the gene expression knockdown activity of the siRNA.

In a preferred embodiment, we describe methods to alter the interaction of an siRNA with a cell or transfection agent comprising: reacting the siRNA with a modifying agent wherein the modifying agent contains a hydrophobic group. The transfection agent can comprise polymers, lipids, detergents, or surfactants, or a combination of polymers, lipids, detergents, or surfactants. Hydrophobic modification of the siRNA allows hydrophobic interaction of the siRNA with the transfection agent. However, because the modifications can add functional groups to siRNA without eliminating charge on the siRNA, the modifications may be made without eliminating the ability to the siRNA to participate in ionic interactions with other molecules, including transfection agents.

In a preferred embodiment, RNA complexes are described comprising: modified RNA/lipid complexes, modified RNA/polymer complexes, and modified RNA/lipid/polymer complexes. Modified RNA/lipid complexes are formed by dissolving the modified RNA in an appropriate organic solvent or in an organic/aqueous solvent mixture and then mixing the modified RNA with lipids or liposomes. The RNA/lipid complex may be applied directly to cells or it may be dried to a film and hydrated with an aqueous solution. Modified RNA/polymer complexes may be formed by mixing the modified siRNA with a polymer or a polymer complex. The modified RNA and polymer may associate through hydrophobic and/or ionic interactions to form the complex. Modification of the RNA, such as with a hydrophobic group, makes it possible to interact the nucleic acid with a polymer via non-ionic interactions. A polymer complex can contain one or more polymers and can contain lipids, surfactants, peptides, and/or proteins. The RNA complexes can additionally possess one or more functional groups selected from the list consisting of: hydrophobic groups, membrane active compounds, cell penetrating compounds, cell targeting signals, interaction modifiers, and steric stabilizers. The functional groups may be associated covalently or non-covalently with the siRNA, lipid, or polyion.

In a preferred embodiment, post-synthetic modification of the RNA increases resistance of the RNA to nucleases. A preferred RNA is an siRNA, microRNA, or other oligonucleotide capable of inhibiting gene expression through RNA interference.

In a preferred embodiment, we describe a process for the delivery of an RNA to a mammalian cell comprising: bringing a modified RNA or modified RNA complex into contact with said cell. The invention is meant to encompass the intravascular delivery of the modified RNA or modified RNA complex to a mammalian cell in vivo. For example, the invention involves diluting the modified RNA or modified RNA complex in an appropriate aqueous solution and injecting the resulting solution into a vessel in the mammal. Alternatively, the modified RNA may be injected into a tissue in the mammal. RNA may also be delivered to a cell in vitro by contacting the cell with the modified RNA or modified RNA complex. A preferred RNA is an siRNA, microRNA, or other oligonucleotide capable of inhibiting gene expression through RNA interference.

Further objects, features, and advantages of the invention will be apparent from the following detailed description when taken in conjunction with the accompanying drawings.

### BRIEF DESCRIPTION OF THE DRAWINGS

FIG. 1. Illustration of an example of silylchloride modification of siRNA.

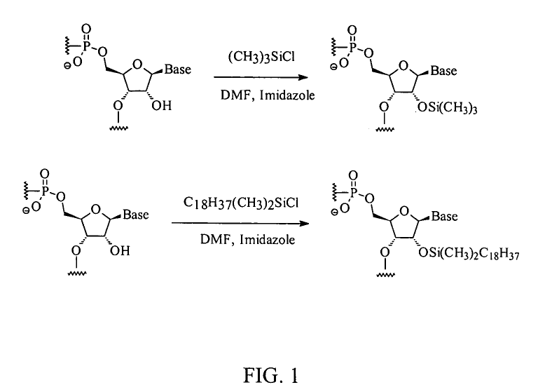

FIG. 2. Illustration of examples of acylation of siRNA.

FIG. 3. Illustration of examples of alkylation of RNA.

FIG. 4. Gel electrophoresis of Amine Modified siRNA exposed to RNAse 1.

FIG. 5. Gel electrophoresis of Hydroxyl Modified siRNA exposed to RNAse 1.

FIG. 6. Flourescent microscopic image of mouse liver tissue section illustrating delivery of modified siRNA to hepatocytes in vivo. (A) Cy3-GL3 siRNA-OLauroyl in hepatocyte nuclei, (B) Phalloidin Alexa 488 stained Actin, (C) ToPro3 stained Nuclei.

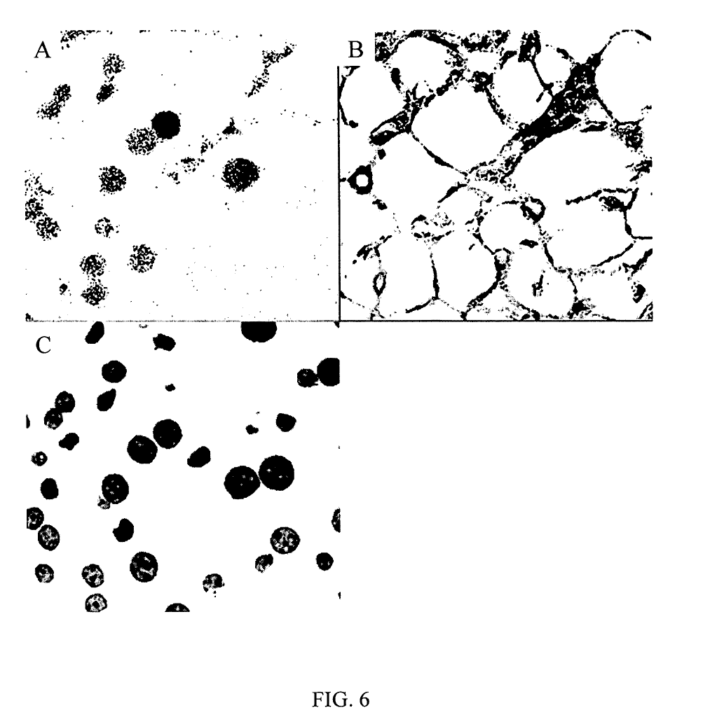

### DETAILED DESCRIPTION OF THE INVENTION

Described are post synthetic covalent modifications of oligonucleotides (siRNA, microRNA, etc.) capable of inducing RNAi in mammalian cells. The modifications can affect the hydrophobicity of the RNA and therefore affect its interactions with cells, proteins, enzymes, lipids, and polymers. The modifications can also impart greater resistance of the RNA to cleavage by nucleases. A stable siRNA has the potential for increased activity or prolonged activity provided the modification does not inactivate the siRNA. The modifications described herein either do not negatively affect siRNA knockdown activity or are reversible. The reversible modifications are labile under physiologically conditions and cleavage of the modification regenerates the original RNA. The resulting modified RNA can be delivered to mammalian cells in vitro and in vivo without further modification or they can be combined with lipid(s) or polymer(s) to enhance delivery of the RNA to the cell.

Covalent modifications of hydroxyl groups are well known to those in the art and encompass a wide range chemical reactions. Examples include, but are not limited to silylation, acylation, and alkylation.

Covalent modification of nitrogen atoms in the nucleotide bases of the RNA, such as the N7 of guanine or the N3 or adenine, is possible using known alkylation agents (U.S. Pat. No. 6,262,252). Additionally, reactions can take place on the phosphate oxygens of nucleic acids to form covalent bonds such as phosphate-amides or phosphate esters. RNA may also be modification through covalent linkage to a hydroxyl group at the 2′ position of the RNA ribose ring. Covalent modification of the RNA hydroxyloxygen can impart greater stability of the RNA molecule to RNAses.

The covalent modification of RNA can be labile in that the covalent bond is cleaved at some point after delivering the sample to the tissue culture (in vitro) or to the animal (in vivo). Cleavage of the labile modification results in the formation of the original RNA molecule.

By synthetic covalent modification, we mean that the RNA has been constructed—synthesized from ribonucleosides or via the degradation of larger RNA—prior to the modification process. The RNA molecule can be single stranded or double stranded, and can be prepared from any natural or synthetic ribonucleoside.

Silylchloride Modification of RNA

In a preferred embodiment, the RNA is modified with a silyl chloride in an appropriate solvent, for example DMF, resulting in silylation of a ribose 2′-OH to form a silyl ether. Silylchlorides are known to react with a wide variety of organic functional groups to yield silylated derivatives [Greene and Wuts 1999]. For example, the reaction of an amine and a silylchloride affords a silazane. The reaction of an alcohol and a silylchloride affords a silyl ether. The amount of silyl chloride in the reaction can be adjusted in order to silylate any number of the hydroxyl groups on a molecule of RNA. From one to all of the hydroxyl groups per RNA molecule may be modified in this manner.

The conditions can also be altered to allow for silylation of other atoms in an RNA. For example, the silyl chloride can react with a phosphate oxygen (resulting in a phosphate silyl ester), a nitrogen in a nucleotide base (resulting in a silazane). Silazanes are generally very hydrolytically labile, and upon hydrolysis, the original amine is regenerated together with a silanol or silyl ether. Phosphate silyl esters are similarly very susceptible to hydrolysis and can hydrolyze back to a phosphate and a silanol. The silyl ether is susceptible to hydrolysis under acidic pH, with the stability dependent on the particular groups bonded to the silicon atom and the steric environment of the ribose [Green T W et al. 1999]. The reaction of the RNA with the silyl chloride can initially take place on a nitrogen and then react on the ribose hydroxyl since the silicon oxygen bond is much stronger (more stable) than the silicon nitrogen bond. Although additional groups on the RNA may be modified by the silyl chloride, for example the phosphate oxygen(s) and the nitrogen bases of the ribose, the RNA in the present invention remains functionally active. Hydrolysis of all silyl chloride modifications results in the regeneration of the original RNA. The silyl chloride can be an alkyl chlorosilane or a bischlorosilane of general formula I. Additionally, the silylating agent can posses additional functionality selected from the list consisting of: hydrophobicity, membrane active compounds, cell penetrating compounds, cell targeting signals, interaction modifiers and steric stabilizers.

The present invention encompasses the modification of RNA with silyl chlorides of general formula I

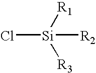

wherein R₁, R₂, and R₃ are independent and are selected from the group consisting of halogen, alkyl, aryl, and substituted alkyl or substituted aryl. More specifically, R₁, R₂, and R₃ are independent and are selected from the group consisting of halogen (chloride or bromide), alkyl (from 1-30 carbons, can contain unsaturation, and can be branched for example in a tert butyl or isopropyl group), aryl (phenyl, or substituted phenyl ring), alkyl chlorosilanes (therefore a bis chlorosilane), membrane active compounds, cell penetrating compounds, cell targeting signals, interaction modifiers, or steric stabilizers.

Acylation of RNA

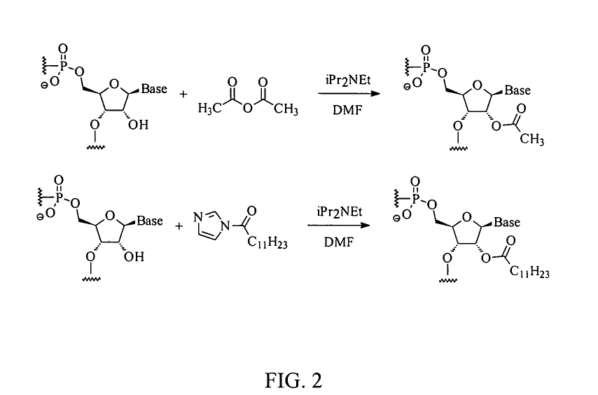

In another preferred embodiment, an RNA is modified with an acylating agent in an appropriate solvent, resulting in the formation of a modified RNA with O-acylation of the 2′ hydroxyl group (esterification of the 2′-OH to form an ester, FIG. 2). Acylation can be controlled by adjusting the reaction conditions and the amount of the acylation agent in the reaction in order to acylate the RNA. As little as a singly hydroxyl per RNA molecule or as many as all of the hydroxyls on an RNA molecule may be acylated. The acylation reaction can be utilized to attach simple groups such as acetyl or more complex systems (longer alkyl chains, ring systems, and heteroatom containing systems). Acyl groups can be hydrolyzed to afford a carboxylic acid and the original RNA. Additionally, acyl groups can be cleaved enzymatically from the RNA.

The nature of the acylating agent depends a variety of conditions, such as the reaction solvent and compatibility with other atoms or functional groups on the molecules. For example if the RNA is dissolved in an organic solvent such as DMF, then the acid chloride or an anhydride of a carboxylic acid can be utilized in the acylation. Additionally, the acylation can be conducted using an activated carboxylic group, for example from the reaction of a carboxylic acid and 1,3-dicyclohexylcarbodiimide (DCC) and 4-(dimethylamino)pyridine (DMAP). Additionally, the acylating agent can possess additional functionality selected from the list consisting of: hydrophobicity, membrane active compounds, cell penetrating compounds, cell targeting signals, interaction modifiers and steric stabilizers.

Alkylation of RNA

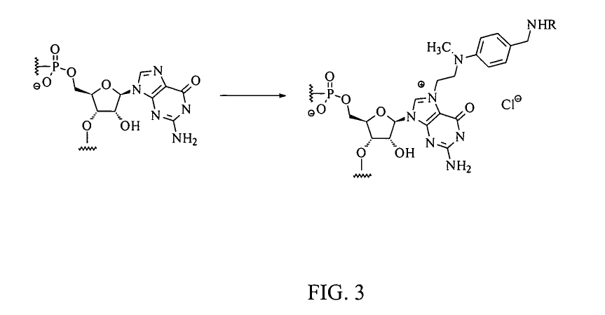

In another preferred embodiment, an RNA can be alkylated with reagents including but not limited to, nitrogen mustards and activated three-membered rings (epoxides, cyclopropanes, episulfides), which possess a pendent group including but not limited to an amine, alkyl group, peptide, carboxylic acid, aldehyde, and ketone (U.S. Pat. No. 6,262,252). FIG. 3 illustrates the N-7 alkylation of a guanine base. As with silylation and acylation, the amount of alkylation can be controlled in order to alkylate varying amounts of the bases.

Modification of Amine-Modified RNA

The alkylating agent may possesses a pendent amine group. The pendent amine group may then be acylated through reversible acylation with compounds derived from maleic anhydrides, for example, 2-propionic-3-methylmaleic anhydride [Naganawa et al. 1994; Hermanson 1996; Reddy and Low 2000; Dinand et al. 2002; Rozema et al. 2003]. The present invention encompasses the reversible modification of amine-modified RNA with compounds of general formula II

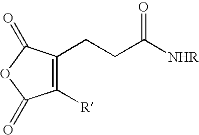

wherein R is selected from the group consisting of: alkyl group (from 1-30 carbons, can contain unsaturation, and can be branched for example in a tert butyl or isopropyl group), aryl group, steric group, and targeting group; and R′ is selected from the group consisting of: hydrogen, alkyl group (from 1-30 carbons, can contain unsaturation, and can be branched for example in a tert butyl or isopropyl group), and aryl group. The resulting modified RNA can be dried, and redissolved in an appropriate organic, aqueous, or mixed solvent.

Maleic anhydrides react with pendent amines on the RNA to form maleamic acids. This reaction is reversible. Maleamic acids are known to be stable under basic conditions, but hydrolyze under acidic conditions. In acidic conditions, the amide bond formed during the reaction between the amine and the anhydride is cleaved to yield the original unmodified amine and the maleic anhydride.

Modified RNA Complexes

The modified RNA may be combined with lipid(s), polymer(s) or a combination of lipid and polymer to form a complex. The RNA modification may facilitate the interaction of the RNA with the lipid or polymer. For example, hydrophobic modification of an RNA can enhance interaction of the RNA with an amphipathic compound through hydrophobic interactions. The RNA complex can then be delivered to the cell for delivery of the RNA to the cell.

The modified RNA may be dissolved in an appropriate organic solvent or in an organic/aqueous solvent mixture, and mixed with lipids to form a modified RNA-lipid complex. The lipid(s) can posses additional functionality selected from the list consisting of: hydrophobic group, membrane active compound, cell penetrating compound, cell targeting signal, interaction modifier and steric stabilizer. Additionally, the lipid(s) can posses reactive groups to which functional groups may be attached.

A modified RNA-lipid complex may be dried to a film. The resulting film is hydrated with an aqueous solution, mixed to form liposomes and applied to cells. The lipid(s) can possess additional functionality, selected from the list consisting of: membrane active compounds, cell penetrating compounds, cell targeting signals, interaction modifiers and steric stabilizers. Additionally, the lipid(s) can posses reactive groups to which functional groups may be attached.

The invention is meant to encompass the delivery of RNA to cells by mixing the modified RNA with lipid(s), or by hydrating lipid(s) with a solution containing the modified RNA to form a modified RNA-lipid complex.

The modified RNA may be mixed with a polymer or a polymer complex resulting in the formation of a modified RNA-polymer complex. The polymer complex can contain one or more polymers and can contain lipids, surfactants, peptides, and/or proteins. Any of the components of the modified RNA-polymer complex can have additional functional groups selected from the list consisting of: hydrophobic groups, membrane active compounds, cell penetrating compounds, cell targeting signals, interaction modifiers and steric stabilizers. The modified RNA-polymer complex is then delivered to cells.

The invention is also meant to encompass the delivery to cells of the modified RNA, modified RNA-lipid complex, or modified RNA-polymer complex via arterial, or venous (intravascular) delivery in vivo. For example, the invention involves diluting the modified RNA, modified RNA-lipid complex, or modified RNA-polymer complex in an appropriate aqueous solution (for example ringers or isotonic glucose) and injecting the resulting solution into the animal.

Definitions

Polynucleotide—The term polynucleotide, or nucleic acid or polynucleic acid, is a term of art that refers to a polymer containing at least two nucleotides. Nucleotides are the monomeric units of polynucleotide polymers. Polynucleotides with less than 120 monomeric units are often called oligonucleotides. Natural nucleic acids have a deoxyribose- or ribose-phosphate backbone. An artificial or synthetic polynucleotide is any polynucleotide that is polymerized in vitro or in a cell free system and contains the same or similar bases but may contain a backbone of a type other than the natural ribose-phosphate backbone. These backbones include: PNAs (peptide nucleic acids), phosphorothioates, phosphorodiamidates, morpholinos, and other variants of the phosphate backbone of native nucleic acids. Bases include purines and pyrimidines, which further include the natural compounds adenine, thymine, guanine, cytosine, uracil, inosine, and natural analogs. Synthetic derivatives of purines and pyrimidines include, but are not limited to, modifications that place new reactive groups such as, but not limited to, amines, alcohols, thiols, carboxylates, and alkylhalides. The term base encompasses any of the known base analogs of DNA and RNA. The term polynucleotide includes deoxyribonucleic acid (DNA) and ribonucleic acid (RNA) and combinations of DNA, RNA and other natural and synthetic nucleotides.

The modifications described herein can be performed on any polynucleotide containing at least one ribose 2′ hydroxyl in the polynucleotide backbone. Therefore, RNA, as used herein, is meant to include any polynucleotide containing at least one nucleotide (base+sugar) with a backbone ribose 2′ hydroxyl group.

A polynucleotide can be delivered to a cell to express an exogenous nucleotide sequence, to inhibit, eliminate, augment, or alter expression of an endogenous nucleotide sequence, or to affect a specific physiological characteristic not naturally associated with the cell.

A polynucleotide-based gene expression inhibitor comprises any polynucleotide containing a sequence whose presence or expression in a cell causes the degradation of or inhibits the function, transcription, or translation of a gene in a sequence-specific manner. Polynucleotide-based expression inhibitors may be selected from the group comprising: siRNA, microRNA, interfering RNA or RNAi, dsRNA, ribozymes, antisense polynucleotides, and DNA expression cassettes encoding siRNA, microRNA, dsRNA, ribozymes or antisense nucleic acids. SiRNA comprises a double stranded structure typically containing 15-50 base pairs and preferably 19-25 base pairs and having a nucleotide sequence identical or nearly identical to an expressed target gene or RNA within the cell. An siRNA may be composed of two annealed polynucleotides or a single polynucleotide that forms a hairpin structure. MicroRNAs (miRNAs) are small noncoding polynucleotides, about 22 nucleotides long, that direct destruction or translational repression of their mRNA targets. Antisense polynucleotides comprise sequence that is complimentary to a gene or mRNA. Antisense polynucleotides include, but are not limited to: morpholinos, 2′-O-methyl polynucleotides, DNA, RNA and the like. The polynucleotide-based expression inhibitor may be polymerized in vitro, recombinant, contain chimeric sequences, or derivatives of these groups. The polynucleotide-based expression inhibitor may contain ribonucleotides, deoxyribonucleotides, synthetic nucleotides, or any suitable combination such that the target RNA and/or gene is inhibited.

Modified RNA—Modified siRNA is siRNA modified on the 2′-OH of the ribose, for example by silylation, acylation, or alkylation. Modified RNA also means RNA alkylated on one or more nitrogen atoms of nucleotides bases in the RNA with a reagent, including but not limited to, nitrogen mustards and activated three-membered rings (epoxides, cyclopropanes, episulfides), which possess a pendent group including but not limited to an amine, alkyl group, peptide, carboxylic acid, aldehyde, and ketone.

Transfection—The process of delivering a polynucleotide to a cell has been commonly termed transfection or the process of transfecting and also it has been termed transformation. The term transfecting as used herein refers to the introduction of a polynucleotide or other biologically active compound into cells. The polynucleotide may be delivered to the cell for research purposes or to produce a change in a cell that can be therapeutic. The delivery of a polynucleotide for therapeutic purposes is commonly called gene therapy. Gene therapy is the purposeful delivery of genetic material to somatic cells for the purpose of treating disease or biomedical investigation. The delivery of a polynucleotide can lead to modification of the genetic material present in the target cell.

Transfection agent—A transfection reagent or delivery vehicle is a compound or compounds that bind(s) to or complex(es) with oligonucleotides and polynucleotides, and mediates their entry into cells. Examples of transfection reagents include, but are not limited to, cationic liposomes and lipids, polyamines, calcium phosphate precipitates, histone proteins, polyethylenimine, polylysine, and polyampholyte complexes. It has been shown that cationic proteins like histones and protamines, or synthetic polymers like polylysine, polyarginine, polyomithine, DEAE dextran, polybrene, and polyethylenimine may be effective intracellular delivery agents. Typically, the transfection reagent has a component with a net positive charge that binds to the oligonucleotide's or polynucleotide's negative charge.

Chemical Bond—A chemical bond is a covalent or noncovalent bond.

Covalent Bond—A covalent bond is a chemical bond in which each atom of the bond contributes one electron to form a pair of electrons. A covalent bond can also mean a coordinate or dative bond.

Noncovalent Bond—A noncovalent bond or ionic bond is a bond in which electrons are transferred to atoms to afford charged atoms. Atoms of opposite charge can form an interaction.

Labile Bond—A labile bond is a covalent bond that is capable of being selectively broken. That is, the labile bond may be broken in the presence of other covalent bonds without the breakage of other covalent bonds. For example, a disulfide bond is capable of being broken in the presence of thiols without cleavage of any other bonds, such as carbon-carbon, carbon-oxygen, carbon-sulfur, carbon-nitrogen bonds, which may also be present in the molecule. Labile also means cleavable.

Labile Linkage—A labile linkage is a chemical compound that contains a labile bond and provides a link or spacer between two other groups. The groups that are linked may be chosen from compounds such as biologically active compounds, membrane active compounds, compounds that inhibit membrane activity, functional reactive groups, monomers, and cell targeting signals.

pH Labile—pH-labile refers to the selective breakage of a covalent bond under acidic conditions (pH<7). That is, the pH-labile bond may be broken under acidic conditions without the breakage of other covalent bonds. The term pH-labile includes both linkages and bonds that are pH-labile, very pH-labile, and extremely pH-labile. A subset of pH-labile bonds is very pH-labile. For the purposes of the present invention, a bond is considered very pH-labile if the half-life for cleavage at pH 5 is less than 45 minutes. A subset of pH-labile bonds is extremely pH-labile. For the purposes of the present invention, a bond is considered extremely pH-labile if the half-life for cleavage at pH 5 is less than 15 minutes.

Mammalian intracellular/extracellular conditions—Mammalian intracellular and extracellular conditions, or physiological conditions, are those physical and chemical conditions which a normally present in a living mammal. Intracellular conditions include the conditions associated with cellular cytoplasm, nuclei, endosomes, lysosomes, etc. Extracellular conditions include conditions associated with the extracellular matrix, serum, and the organ lumena.

Hydrophobation—Hydrophobation, or hydrophobic modification, is the act of associating a compound that possesses a hydrophobic group, such as a surfactant, with another compound via a chemical bond.

Amphiphilic and Amphipathic Compounds—Amphipathic, or amphiphilic, compounds have both hydrophilic (water-soluble) and hydrophobic (water-insoluble) parts. Amphipathic compounds include polymers containing pendent hydrophobic groups, natural and synthetic lipids, steroids, fatty acids, surfactants, and detergents.

Surfactant—A surfactant is a surface active agent, such as a detergent or a lipid, which is added to a liquid to increase its spreading or wetting properties by reducing its surface tension. A surfactant refers to a compound that contains a polar group (hydrophilic) and a non-polar (hydrophobic) group on the same molecule. A cleavable surfactant is a surfactant in which the polar group may be separated from the nonpolar group by the breakage or cleavage of a chemical bond located between the two groups, or to a surfactant in which the polar or non-polar group or both may be chemically modified such that the detergent properties of the surfactant are destroyed.

Micelle—Micelles are microscopic vesicles that contain amphipathic molecules but do not contain an aqueous volume that is entirely enclosed by a membrane. In micelles the hydrophilic part of the amphipathic compound is on the outside (on the surface of the vesicle). In inverse micelles the hydrophobic part of the amphipathic compound is on the outside. The inverse micelles thus contain a polar core that can solubilize both water and macromolecules within the inverse micelle.

Liposome—Liposomes are microscopic vesicles that contain bilayers of amphipathic molecules and typically contain an aqueous volume that is entirely enclosed by a membrane.

Microemulsions—Microemulsions are isotropic, thermodynamically stable solutions in which substantial amounts of two immiscible liquids (water and oil) are brought into a single phase due to a surfactant or mixture of surfactants. The spontaneously formed colloidal particles are globular droplets of the minor solvent, surrounded by a monolayer of surfactant molecules. The spontaneous curvature, H0 of the surfactant monolayer at the oil/water interface dictates the phase behavior and microstructure of the vesicle. Hydrophilic surfactants produce oil in water (O/W) microemulsions (H0>0), whereas lipophilic surfactants produce water in oil (W/O) microemulsions.

Drying—Drying means removing the solvent from a sample, for example, removing the solvent from a complex under reduced pressure. Drying also means dehydrating a sample, or lyophilizing of a sample.

Salt—A salt is any compound containing ionic bonds; i.e., bonds in which one or more electrons are transferred completely from one atom to another. Salts are ionic compounds that dissociate into cations and anions when dissolved in solution and thus increase the ionic strength of a solution. Pharmaceutically acceptable salt means both acid and base addition salts. A pharmaceutically acceptable acid addition salt is a salt that retains the biological effectiveness and properties of the free base, is not biologically or otherwise undesirable, and is formed with inorganic acids and organic acids. A pharmaceutically acceptable base addition salt is a salt that retains the biological effectiveness and properties of the free acid, is not biologically or otherwise undesirable, and is prepared from the addition of an inorganic organic base to the free acid.

Functional group—Functional groups include cell targeting signals, membrane active compounds, hydrophobic groups, cell penetrating compounds, and other compounds that alter the behavior or interactions of the compound or complex to which they are attached. Additionally, a functional group also means a chemical functional group that can undergo further chemical reactions. Examples include but are not limited to hydroxyl groups, amine groups, thiols, carboxylic acids, aldehydes, and ketones.

Targeting groups—Targeting groups, or ligands, are used for targeting a molecule or complex to cells, to specific cells, to tissues or to specific locations in a cell. Targeting groups enhance the association of molecules with a cell. Examples of targeting groups include those that target to the asialoglycoprotein receptor by using asialoglycoproteins or galactose residues. Other proteins such as insulin, EGF, or transferrin can be used for targeting. Other targeting groups include molecules that interact with membranes such as fatty acids, cholesterol, dansyl compounds, and amphotericin derivatives. A variety of ligands have been used to target drugs and genes to cells and to specific cellular receptors. The ligand may seek a target within the cell membrane, on the cell membrane or near a cell. Binding of a ligand to a receptor may initiate endocytosis. Nuclear localization signals are examples of targeting groups that enhance localization of molecules to specific subcellular locations.

Membrane active compound—Membrane active polymers or compounds are molecules that are able to inducing one or more of the following effects upon a biological membrane: an alteration that allows small molecule permeability, pore formation in the membrane, a fusion and/or fission of membranes, an alteration that allows large molecule permeability, or a dissolving of the membrane. This alteration can be functionally defined by the compound's activity in at least one the following assays: red blood cell lysis (hemolysis), liposome leakage, liposome fusion, cell fusion, cell lysis and endosomal release. More specifically membrane active compounds allow for the transport of molecules with molecular weight greater than 50 atomic mass units to cross a membrane. This transport may be accomplished by either the loss of membrane structure or the formation of holes or pores in the membrane. Membrane active polymers may be selected from the list comprising: membrane active toxins such as pardaxin, melittin, cecropin, magainin, PGLa, indolicidin, and dermaseptin; synthetic amphipathic peptides; and amphipathic polymers such as butyl polyvinyl ether. There exists little to no homology or structural similarity between all the different membrane active peptides. Therefore, they are defined by their membrane activity.

Cell penetrating compounds—Cell penetrating compounds, which include cationic import peptides (also called peptide translocation domains, membrane translocation peptides, arginine-rich motifs, cell-penetrating peptides, and peptoid molecular transporters) are typically rich in arginine and lysine residues and are capable of crossing biological membranes. In addition, they are capable of transporting molecules to which they are attached across membranes. Examples include TAT (GRKKRRQRRR, SEQ ID 9), VP22 peptide, and an ANTp peptide (RQIKIWFQNRRMKWKK, SEQ ID 10). Cell penetrating compounds are not strictly peptides. Short, non-peptide polymers that are rich in amines or guanidinium groups are also capable of carrying molecules crossing biological membranes. Like membrane active peptides, cationic import peptides are defined by their activity rather than by strict amino acid sequence requirements.

Interaction Modifiers—An interaction modifier changes the way that a molecule interacts with itself or other molecules relative to molecule containing no interaction modifier. The result of this modification is that self-interactions or interactions with other molecules are either increased or decreased. Polyethylene glycol is an interaction modifier that decreases interactions between molecules and themselves and with other molecules.

Steric Stabilizer—A steric stabilizer is a long chain hydrophilic group that prevents aggregation by sterically hindering particle to particle or polymer to polymer electrostatic interactions. Examples include: alkyl groups, PEG chains, polysaccharides, alkyl amines. Electrostatic interactions are the non-covalent association of two or more substances due to attractive forces between positive and negative charges.

Chelator—A Chelator is a polydentate ligand, a molecule that can occupy more than one site in the coordination sphere of an ion, particularly a metal ion, primary amine, or single proton. Examples of chelators include crown ethers, cryptates, and non-cyclic polydentate molecules. A crown ether is a cyclic polyether containing (—X—(CR1-2)n)m units, where n=1-3 and m=3-8. The X and CR1-2 moieties can be substituted, or at a different oxidation states. X can be oxygen, nitrogen, or sulfur, carbon, phosphorous or any combination thereof. R can be H, C, O, S, N, P. The crown ether ring system is named as [(n+1)m crown m] for X=oxygen, as [(n+1)m azacrown m] when X=nitrogen, as [(n+1)m thiocrown m] when X=sulfur. In the case of two or more heteroatoms present in the ring the heteroatom positions are specified. A subset of crown ethers described as a cryptate contain a second (—X—(C_{R1}-2)n)_{z} strand where z=3-8. The beginning X atom of the strand is an X atom in the (—X—(C_{R1}-2)n)ₘ unit, and the terminal 5CH₂ of the new strand is bonded to a second X atom in the (—X—(C_{R1}-2)n)ₘ unit. Non-cyclic polydentate molecules containing (—X—(C_{R1}-2)n)ₘ unit(s), where n=1-4 and m=1-8. The X and C_{R1-2} moieties can be substituted, or at a different oxidation states. X can be oxygen, nitrogen, or sulfur, carbon, phosphorous or any combination thereof.

Polymer—A polymer is a molecule built up by repetitive bonding together of smaller units called monomers. A polymer can be linear, branched network, star, comb, or ladder types of polymer. A polymer can be a homopolymer in which a single monomer is used or can be copolymer in which two or more monomers are used. The main chain of a polymer is composed of the atoms whose bonds are required for propagation of polymer length. For example in poly-L-lysine, the carbonyl carbon, α-carbon, and α-amine groups are required for the length of the polymer and are therefore main chain atoms. The side chain of a polymer is composed of the atoms whose bonds are not required for propagation of polymer length.

To those skilled in the art of polymerization, there are several categories of polymerization processes that can be utilized in the described process. The polymerization can be chain or step. Template polymerization can be used to form polymers from daughter polymers.

Other Components of the Monomers and Polymers: Polymers may have functional groups that enhance their utility. These groups can be incorporated into monomers prior to polymer formation or attached to the polymer after its formation. Functional groups may be selected from the list consisting of: targeting groups, interaction modifiers, steric stabilizers, and membrane active compounds, and affinity groups.

Polyion—A polyion (or polyelectrolyte), is a polymer possessing charge, i.e. the polymer contains a group (or groups) that has either gained or lost one or more electrons. The term polyion includes polycations, polyanions, zwitterionic polymers, and neutral polymers. The term zwitterionic refers to the product (salt) of the reaction between an acidic group and a basic group that are part of the same molecule. Salts are ionic compounds that dissociate into cations and anions when dissolved in solution. Salts increase the ionic strength of a solution, and consequently decrease interactions between nucleic acids with other cations. A charged polymer is a polymer that contains residues, monomers, groups, or parts with a positive or negative charge and whose net charge can be neutral, positive, or negative.

Polycation—A polycation can be a polymer possessing net positive charge. A polymeric polycation can contain monomer units that are charge positive, charge neutral, or charge negative, however, the net charge of the polymer must be positive. A polycation also can be a non-polymeric molecule that contains two or more positive charges.

Polyanion—A polyanion can be a polymer containing a net negative charge. A polymeric polyanion can contain monomer units that are charge negative, charge neutral, or charge positive, however, the net charge on the polymer must be negative. A polyanion can also be a non-polymeric molecule that contains two or more negative charges.

Delivery—Delivery of a polynucleotide means to transfer the polynucleotide from a container outside a mammal to near or within the outer cell membrane of a cell in the mammal. The term transfection is used herein, in general, as a substitute for the term delivery, or, more specifically, the transfer of a polynucleotide from directly outside a cell membrane to within the cell membrane. Parenteral routes of administration include intravascular (intravenous, intra-arterial), intramuscular, intraparenchymal, intradermal, subdermal, subcutaneous, intratumor, intraperitoneal, intrathecal, subdural, epidural, and intralymphatic injections that use a syringe and a needle or catheter. Intravascular herein means within a tubular structure called a vessel that is connected to a tissue or organ within the body. Within the cavity of the tubular structure, a bodily fluid flows to or from the body part. Examples of vessels include arteries, arterioles, capillaries, venules, sinusoids, veins, lymphatics, and bile ducts. An administration route involving the mucosal membranes is meant to include nasal, bronchial, inhalation into the lungs, or via the eyes. Transdermal routes of administration have been effected by patches and ionotophoresis. Other epithelial routes include oral, nasal, respiratory, and vaginal routes of administration.

### EXAMPLES

### Example 1

### Silylation of GL2 RNA

Part A. Silylation of dsRNA with Chlorotrimethyl Silane. To 2.0 μg of annealed ds RNA GL-2 siRNA (20 μL of a 100 ng/μL solution in water, 150 pmol dsRNA, 0.0063 μmol —OH, 2′OH-CGUA-CGCGGAAUACUUCGAdTdT (SEQ ID 1) and its compliment 2′OH-UCGAAGUAUUCC-GCGUACGdTdT, (SEQ ID 2), TriLink BioTechnologies Inc.) was added 60 μL of anhydrous dimethylformamide (Aldrich Chemical Company). To the resulting solution was added chlorotrimethylsilane (10 μL of a 0.1 mg/mL solution in DMF, 0.011 mmol, Aldrich Chemical Company), and diisopropylethylamine (1.9 μL, 0.011 mmol, Aldrich Chemical Company). The solution was stirred for 4 hrs to afford GL2-OTMS. After 4 hrs, sample was placed into 10 vials for formulation (200 ng per vial).

Part B. Silylation of dsRNA with Chlorodimethyloctadecylsilane. To 2.0 μg of annealed GL-2 siRNA (40 μL of a 50 ng/μL solution in water, 150 pmol dsRNA, 0.0063 μmol —OH, SEQ ID 1 and its compliment SEQ ID 2, TriLink BioTechnologies Inc.) was added 100 μL of anhydrous dimethylformamide (Aldrich Chemical Company). To the resulting solution was added chlorodimethyloctadecylsilane (2.0 mg, 0.0058 mmol, Aldrich Chemical Company), and diisopropylethylamine (1 μL, 0.0058 mmol, Aldrich Chemical Company). The solution was stirred for 4 hrs to afford GL2-OSiC18. After 4 hrs, 860 μL of 150 mM NaCl was added to the sample, and sample was placed into 10 vials for formulation (200 ng per vial). Rinsed with 200 μL of EtOH, which was added to the 10 vials (20 μL each). An additional 80 μL of 150 mM NaCl was added to each well, to bring the total volume of the samples to 200 μL.

Part C. Transfection of 3T3-Luc Cells. Delivery of GL2 siRNA to 3T3-Luc cells results in knockdown of expression of the luciferase gene present in these cells. Samples were prepared from GL2-OTMS (Part A) and GL2-OSiC18 (Part B). For GL2-OTMS, 150 mM NaCl was added to each tube to bring the volume to 200 μL. The modified siRNAs were then combined with the transfections agents: TransIT-TKO, MC789 (a lipid), TransIT LT-1 (polymer/lipid formulation), and PD (polymer formulation). Transfections were conducted in duplicate in 12 well plates by covering the cells with 500 μL DMEM with 10% serum and adding 100 μL of transfection sample. Cells were harvested 24 hr post transfection, and read on a luminometer. RLUs are the average of the two wells.

| siRNA | transfection | Mean | | |
|---|---|---|---|---|
| (200 ng) | agent | RLU | % Expression | Confluency |
| blank | — | 1,120,831 | 100 | 100 |
| GL2 | — | 1,138,549 | 102 | 100 |
| GL2 | 4 μl TKO | 807,490 | 72 | 100 |
| GL2 | 4 μl LT1 | 1,569,163 | 104 | 100 |
| GL2 | 200 ng PD | 605,005 | 54 | 100 |
| GL2-OTMS | — | 455,443 | 41 | 100 |
| GL2-OTMS | 4 μl TKO | 440,821 | 39 | 100 |
| GL2-OTMS | 8 μl TKO | 394,904 | 35 | 100 |
| GL2-OTMS | 3 μg MC798 | 495,931 | 44 | 100 |
| GL2-OTMS | 6 μg MC798 | 498,063 | 44 | 100 |
| GL2-OTMS | 4 μl LT1 | 458,368 | 41 | 100 |
| GL2-OTMS | 8 μl LT1 | 437,507 | 39 | 100 |
| GL2-OTMS | 100 ng PD | 424,369 | 38 | 100 |
| GL2-OTMS | 200 ng PD | 426,438 | 38 | 100 |
| GL2-OTMS | 100 ng PD/ | 312,033 | 28 | 100 |
| | 4 μl LT1 | | | |
| GL2-OSiC18 | — | 453,046 | 40 | 85 |
| GL2-OSiC18 | 4 μl TKO | 562,939 | 50 | 85 |
| GL2-OSiC18 | 8 μl TKO | 408,958 | 36 | 80 |
| GL2-OSiC18 | 3 μg MC798 | 144,346 | 13 | 85 |
| GL2-OSiC18 | 6 μg MC798 | 334,974 | 30 | 85 |
| GL2-OSiC18 | 4 μl LT1 | 307,985 | 27 | 95 |
| GL2-OSiC18 | 8 μl LT1 | 389,550 | 35 | 95 |
| GL2-OSiC18 | 100 ng PD | 364,363 | 33 | 95 |
| GL2-OSiC18 | 200 ng PD | 566,588 | 51 | 90 |
| GL2-OSiC18 | 100 ng PD/ | 229,213 | 20 | 100 |
| | 4 μl LT1 | | | |

The results demonstrate that directly applying the modified siRNA to cells, in the absence of transfection agents, results in delivery of the siRNA to the cells and gene knockdown by the siRNA. This result is in contrast to the unmodified siRNA, which shows no delivery or knockdown of gene expression in the absence of a transfection reagent. In addition, delivery of siRNA to cells with the transfection agents in generally improved. These results also demonstrate that these modification do not inactivate the delivered siRNA. Nor do these modifications cause cellular toxicity.

### Example 2

### Acylation of GL2 RNA

Part A. Acylation with Acetic Anhydride. To 2.0 μg of annealed GL2 siRNA (40 μL of a 50 ng/μL solution in water, 150 pmol dsRNA, 0.0063 μmol —OH) was added 160 μL of anhydrous dimethylformamide (Aldrich Chemical Company). To this solution was added acetic anhydride (1.3 μg, 0.013 μmol, Aldrich Chemical Company), followed by diisopropylethylamine (0.81 μg, 0.0063 μmol, Aldrich Chemical Company), and the solution was stirred at RT for 4 hr to afford GL2-OAc.

Part B. Acylation with Lauroylimidazole. To 2.0 μg of annealed GL2 siRNA (40 μL of a 50 ng/μL solution in water, 150 pmol dsRNA, 0.0063 μmol —OH) was added 160 μL of anhydrous dimethylformamide (Aldrich Chemical Company). To this solution was added a DMF solution (10 μL) of lauroyl chloride (1.4 μg, 0.0063 μmol, Aldrich Chemical Company) and imidazole (2.1 μg, 0.032 μmol, Aldrich Chemical Company). The resulting solution was stirred at RT for 4 hr to afford GL2-OLauroyl.

Part C. Transfection of 3T3-Luc Cells. Delivery of GL2 siRNA to 3T3-Luc cells results in knockdown of expression of the luciferase gene present in these cells. Complexes for transfection were prepared in 200 μL 150 mM NaCl, for transfection of 3T3-Luc Cells in 12 well plates. Transfections were conducted in duplicate in 12 well plates by covering the cells with 500 μL DMEM with 10% serum and adding 100 μL of transfection sample. Cells were harvested 24 hr post transfection, and read on a luminometer. RLUs are the mean of the two wells. Transfection samples were prepared using TransIT-TKO, MC789 (a lipid), TransIT LT-1 (polymer/lipid formulation), and PD (cationic polymer formulation) transfection agents.

| siRNA | | Mean | % | |
|---|---|---|---|---|
| (200 ng) | transfection agent | RLU | Expression | Confluency |
| blank | — | 723,394 | 100 | 100 |
| GL2 | — | 656,284 | 91 | 100 |
| GL2 | 6 μl TKO | 419,717 | 58 | 100 |
| GL2 | 4 μl LT1 | 687,224 | 95 | 100 |
| GL2 | 200 ng PD/ | 553,131 | 76 | 100 |
| | 4 μg MC798 | | | |
| GL2-OAc | — | 186,966 | 26 | 100 |
| GL2-OAc | 4 μl TKO | 117,781 | 16 | 98 |
| GL2-OAc | 8 μl TKO | 41,289 | 6 | 98 |
| GL2-OAc | 3 μg MC798 | 179,181 | 25 | 98 |
| GL2-OAc | 6 μg MC798 | 176,961 | 24 | 98 |
| GL2-OAc | 4 μl LT1 | 203,417 | 28 | 98 |
| GL2-OAc | 8 μl LT1 | 158,702 | 22 | 98 |
| GL2-OAc | 200 ng PD/ | 151,685 | 21 | 95 |
| | 4 μg MC798 | | | |
| GL2-OAc | 400 ng PD/ | 156,054 | 22 | 95 |
| | 4 μg MC798 | | | |
| GL2-OAc | 200 ng PD | 205,231 | 28 | 85 |
| GL2-OLauroyl | — | 231,177 | 32 | 95 |
| GL2-OLauroyl | 4 μl TKO | 98,656 | 14 | 98 |
| GL2-OLauroyl | 8 μl TKO | 35,537 | 5 | 88 |
| GL2-OLauroyl | 3 μg MC798 | 170,228 | 24 | 95 |
| GL2-OLauroyl | 6 μg MC798 | 130,450 | 18 | 95 |
| GL2-OLauroyl | 4 μl LT1 | 190,919 | 26 | 95 |
| GL2-OLauroyl | 8 μl LT1 | 221,013 | 31 | 95 |
| GL2-OLauroyl | 200 ng PD/ | 114,833 | 16 | 95 |
| | 4 μg MC798 | | | |
| GL2-OLauroyl | 400 ng PD/ | 192,943 | 27 | 93 |
| | 4 μg MC798 | | | |
| GL2-OLauroyl | 200 ng PD | 199,786 | 28 | 93 |

The results demonstrate that directly applying the modified siRNA to cells, in the absence of transfection agent, results in delivery of the siRNA to the cells and gene knockdown by the siRNA. This result is in contrast to the unmodified siRNA, which shows no delivery or 5 knockdown of gene expression in the absence of a transfection reagent. In addition, delivery of siRNA to cells with several transfection agents is improved. These results also demonstrate that these modification do not inactivate the delivered siRNA. Nor do these modifications cause cellular toxicity.

### Example 3

### Acylation of GL3 RNA

GL3 siRNA (2′OH-CWUACGCUGAGUAC-UUCGAdTdT (SEQ ID 3) and its compliment 2′OH-UCGAAGUACUCAGCGUAAGdTdT (SEQ ID 4) was acylated as described in example 2 to afford GL3-OAc and GL3-OLauroyl.

Transfection of CHO-Luc Cells. Delivery of GL3 siRNA to CHO-Luc cells results in knockdown of expression of the luciferase gene present in these cells. Complexes for transfection were prepared in 200 μL of 150 mM NaCl, for transfection of CHO-Luc Cells in 12 well plates. Transfections were conducted in duplicate in 12 well plates by covering the cells with 500 μL DMEM with 10% serum and adding 100 μL of transfection sample. Cells were harvested 24 hr post transfection, and read on a luminometer. RLUs are the mean of the two wells. Transfection samples were prepared using TransIT-TKO, MC789 (a lipid), TransIT LT-1 (polymer/lipid formulation), and PD (cationic polymer formulation) transfection agents.

| siRNA | | Mean | % | |
|---|---|---|---|---|
| (200 ng) | transfection agent | RLU | Expression | Confluency |
| none | — | 444,579 | 100 | 80 |
| GL3 | — | 398,290 | 90 | 80 |
| GL3 | 6 μl TKO | 95,351 | 21 | 70 |
| GL3 | 4 μl LT1 | 502,374 | 113 | 80 |
| GL3 | 200 ng PD/ | 380,435 | 86 | 75 |
| | 4 μg MC798 | | | |
| GL3-OAc | — | 463,545 | 104 | 60 |
| GL3-OAc | 4 μl TKO | 163,990 | 37 | 55 |
| GL3-OAc | 8 μl TKO | 62,937 | 14 | 55 |
| GL3-OAc | 3 μg MC798 | 376,053 | 85 | 60 |
| GL3-OAc | 6 μg MC798 | 418,086 | 94 | 65 |
| GL3-OAc | 4 μl LT1 | 214,367 | 48 | 70 |
| GL3-OAc | 8 μl LT1 | 141,334 | 32 | 70 |
| GL3-OAc | 200 ng PD/ | 287,555 | 65 | 70 |
| | 4 μg MC798 | | | |
| GL3-OAc | 400 ng PD/ | 246,231 | 55 | 60 |
| | 4 μg MC798 | | | |
| GL3-OLauroyl | — | 480,119 | 108 | 55 |
| GL3-OLauroyl | 4 μl TKO | 107,082 | 24 | 55 |
| GL3-OLauroyl | 8 μl TKO | 76,737 | 17 | 55 |

The results demonstrate that these modifications do not disrupt GL3 activity in inhibiting gene expression. When delivered with the TransIT-TKO transfection agent, there was no observable loss of gene knockdown activity with the modified siRNAs. When delivered with the LT1 and PD/MC798 transfection agents, there was increased delivery of the siRNA to the cells.

### Example 4

### In Vivo Delivery of Modified siRNAs and Gene Expression Knockdown

Several complexes were prepared. All complexes contained 10 ml Ringer's to which pGL3-control (a plasmid with a SV40 promoter driving the Firefly Luciferase expression cassette, 20 μL of 2 μg/RL solution in water), and pRLSV40 (a plasmid with a SV40 promoter driving the Renilla Luciferase expression cassette, 2 μL of 2 μg/μL solution in water) was added. In addition, the following were added:

Complex I. No additional components.

Complex II. 20 μg unmodified GL3 siRNA (1.5 μL of 13.3 μg/μL solution).

Complex III. 20 μg unmodified EGFP siRNA (5′ GACGUAAACGGCCACAAGUGC 3′ (SEQ ID 5) and it's compliment 3′CGCUGCAUWUGCCGGUGUUCA 5′, (SEQ ID 6), 1.5 μL of 13.3 μg/μL solution).

Complex IV. 20 μg GL3-OAc siRNA, prepared by diluting 20 μg of GL3 siRNA into 100 μL of DMF, and treating with 6.3 μmol of acetic anhydride (100 eq based on 2′-OH), and 6.3 μmol of diisoropylethylamine (100 eq based on 2′-OH) for 4 hrs.

Complex V. 20 μg EGFP-OAc siRNA, prepared by diluting 20 μg of EGFP siRNA into 100 μL of DMF, and treating with 6.3 μmol of acetic anhydride (100 eq based on 2′-OH), and 6.3 μmol of diisoropylethylamine (100 eq based on 2′-OH) for 4 hrs.

Complex VI. 20 μg GL3-OLauroyl siRNA, prepared by diluting 20 μg GL3 into 100 μL DMF and treating with 3.2 μmol of lauroyl chloride (50 eq based on 2′-OH), 6.3 μmol imidazole (100 eq based on 2′-OH), 3.2 μmol diisoropylethylamine (50 eq based on 2′-OH) for 4 h.

Complex VII. 20 μg EGFP-OLauroyl siRNA, prepared by diluting 20 μg EGFP siRNA into 100 μL of DMF, and treating with 3.2 μmol lauroyl chloride (50 eq based on 2′-OH), 6.3 μmol imidazole (100 eq based on 2′-OH), and 3.2 μmol of diisoropylethylamine (50 eq based on 2′-OH) for 4 hrs.

Complex VIII. 20 μg GL3-OTMS siRNA, prepared by diluting 20 μg of GL3 into 100 μL of DMF, and treating with 6.3 μmol of trimethylsilyl chloride (100 eq based on 2′-OH), and 6.3 μmol of diisoropylethylamine (100 eq based on 2′-OH) for 4 hrs.

Complex IX. 20 μg EGFP-OTMS siRNA, prepared by diluting 20 μg of EGFP siRNA into 100 μL of DMF, and treating with 6.3 μmol of trimethylsilyl chloride (100 eq based on 2′-OH), and 6.3 μmol of diisoropylethylamine (100 eq based on 2′-OH) for 4 hrs.

2.5 mL tail vein injections of 2.5 mL of the complex were preformed on ICR mice (n=3) using a 30 gauge, 0.5 inch needle [Zhang et al 1999]. One day after injection, the animal was sacrificed, and a dual luciferase assay was conducted on the liver. Luciferase and Renilla expression was determined on a Centro LB960 plate luminometer (Berthold Technologies).

| siRNA | Firefly Luc | Renilla | Luc/Ren |
|---|---|---|---|
| none | 94959743 | 73016643 | 133 |
| GL3 | 12887103 | 107423763 | 12 |
| EGFP | 100792687 | 73178893 | 140 |
| GL3-OAc | 12374050 | 165455010 | 8 |
| EGFP-OAc | 113926383 | 99152960 | 117 |
| GL3-OLauroyl | 18595410 | 130123423 | 14 |
| EGFP-OLauroyl | 90218713 | 111691040 | 82 |
| GL3-OTMS | 15094120 | 138786530 | 10 |
| EGFP-OTMS | 96866017 | 258268517 | 37 |

The results demonstrate that the modified siRNAs are effective for gene specific expression knockdown when delivered to cells in vivo.

### Example 5

### Amine Modification of siRNAs with Label-IT Amine

Synthesis of MC998: GL3-NH₂ (5 eq). To H₂O (41.2 mL) was added GL3 siRNA (100 μg, 58.8 μL of 1.7 μg/μL, 7.5 nmol). Label-IT Amine (10 μg, 1.0 μL of 10 μg/μL DMSO, 38 nmol, Mirus Corporation) was added and vortexed followed by the addition of 1N NaOH (0.4 μL). The reaction was incubated at 37° C. for 1 hr. The reaction was removed from heat. After the reaction reached ambient temperature, the siRNA was ethanol precipitated.

Synthesis of MC1002: GL3-NH₂ (21 eq). To H₂O (41.2 μL) was added GL3 siRNA (100 μg, 58.8 μL of 1.7 μg/μL, 7.5 nmol) and gently mixed. Label-IT Amine (43 μg, 4.3 μL of 10 μg/μL DMSO, 160 nmol) was added and vortexed followed by the addition of 1N NaOH (0.4 μL). The reaction was incubated at 37° C. for 1 hr. The reaction was removed from heat. After the reaction reached ambient temperature, the siRNA was ethanol precipitated.

Synthesis of MC1006: EGFP-NH₂ (5 eq). To H₂O (92.5 μL) was added EGFP siRNA (100 μg, 7.5 μL of 13.4 μg/μL, 7.5 nmol) and gently mixed. Label-IT Amine (10.2 μg, 1.0 μL of 10 μg/μL DMSO, 38 nmol) was added and vortexed followed by the addition of 1N NaOH (0.4 μL). The reaction was incubated at 37° C. for 1 hr. The reaction was removed from heat. After the reaction reached ambient temperature, the siRNA was ethanol precipitated.

Synthesis of MC1010: EGFP NH₂ (21 eq). To H₂O (92.5 μL) was added EGFP siRNA (100 μg, 7.5 μL of 13.4 μg/μL, 7.5 nmol) and gently mixed. Label-IT Amine (43 μg, 4.3 μL of 10 μg/μL DMSO, 160 nmol) was added and vortexed followed by the addition of 1N NaOH (0.4 μL). The reaction was incubated at 37° C. for 1 hr. The reaction was removed from heat. After the reaction reached ambient temperature, the siRNA was ethanol precipitated.

Each modified siRNA was brought up in 50 μL H₂O (2 μg/μL) and stored at −20° C.

### Example 6

### Modification of siRNA-NH₂ with NHS-PEG. Primary Amine-Modified siRNAs (MC998, MC1002, MC1006, and MC101) were Acylated with Acylating Agents

Synthesis of MC999: To 0.1M sodium phosphate buffer pH 7.4 (12.5 μL) was added MC998 (25 μg, 12.5 μL of 2 μg/μL H₂O, 8.6 nmol) and vortexed. mPegSPA 5k (43 μg, 0.86 μL of 50 μg/μL DMSO, 8.6 nmol, Shearwater Chemical) was added to the siRNA and vortexed. The reaction was shaken for 1 hr at RT, followed by ethanol precipitation.

Synthesis of MC 1000: To 0.1 M sodium phosphate buffer pH 7.4 (12.5 μL) was added MC998 (25 μg, 12.5 μL of 2 μg/μL H₂O, 8.6 nmol) and vortexed. mPeg₂NHS 10k (86 μg, 1.7 μL of 50 μg/μL DMSO, 8.6 nmol, Shearwater Chemical) was added to the siRNA and vortexed. The reaction was shaken for 1 hr at RT, followed by ethanol precipitation.

Synthesis of MC1001: To 0.1 M sodium phosphate buffer pH 7.4 (12.5 μL) was added MC998 (25 μg, 12.5 μL of 2 μg/μL H₂O, 8.6 nmol) and vortexed. mPeg₂NHS 20k (170 μg, 3.4 μL of 50 μg/μL DMSO, 8.6 nmol, Shearwater Chemical) was added to the siRNA and vortexed. The reaction was shaken for 1 hr at RT, followed by ethanol precipitation.

Synthesis of MC1003: To 0.1M sodium phosphate buffer pH 7.4 (12.5 μL) was added MC1002 (25 μg, 12.5 μL of 2 μg/μL H₂O, 29 nmol) and vortexed. mPegSPA 5k (140 μg, 2.8 μL of 50 μg/μL DMSO, 29 nmol, Shearwater Chemical) was added to the siRNA and vortexed. The reaction was shaken for 1 hr at RT, followed by ethanol precipitation.

Synthesis of MC1004: To 0.1M sodium phosphate buffer pH 7.4 (12.5 μL) was added MC1002 (25 μg, 12.5 μL of 2 μg/μL H₂O, 29 nmol) and vortexed. mPeg₂NHS 10k (290 μg, 5.8 μL of 50 μg/μL DMSO, 29 nmol, Shearwater Chemical) was added to the siRNA and vortexed. The reaction was shaken for 1 hr at RT, followed by ethanol precipitation. Synthesis of MC1005: To 0.1M sodium phosphate buffer pH 7.4 (12.5 μL) was added MC1002 (25 μg, 12.5 μL of 2 μg/μL H₂O, 29 nmol) and vortexed. mPeg₂NHS 20k (580 μg, 11.6 μL of 50 μg/μL DMSO, 29 nmol, Shearwater Chemical) was added to the siRNA and vortexed. The reaction was shaken for 1 hr at RT, followed by ethanol precipitation.

Synthesis of MC1007: To 0.1 M sodium phosphate buffer pH 7.4 (12.5 μL) was added MC1006 (25 μg, 12.5 μL of 2 μg/μL H₂O, 8.6 nmol) and vortexed. mPegSPA 5k (43 μg, 0.86 μL of 50 μg/μL DMSO, 8.6 nmol, Shearwater Chemical) was added to the siRNA and vortexed. The reaction was shaken for 1 hr at RT, followed by ethanol precipitation.

Synthesis of MC1008: To 0.1 M sodium phosphate buffer pH 7.4 (12.5 μL) was added MC1006 (25 μg, 12.5 μL of 2 μg/μL H₂O, 8.6 nmol) and vortexed. mPeg₂NHS 10k (86 μg, 1.7 μL of 50 μg/μL DMSO, 8.6 nmol, Shearwater Chemical) was added to the siRNA and vortexed. The reaction was shaken for 1 hr at RT, followed by ethanol precipitation.

Synthesis of MC1009: To 0.1 M sodium phosphate buffer pH 7.4 (12.5 μL) was added MC1006 (25 μg, 12.5 μL of 2 μg/μL H₂O, 8.6 nmol) and vortexed. mPeg₂NHS 20k (170 μg, 3.4 μL of 50 μg/μL DMSO, 8.6 nmol, Shearwater Chemical) was added to the siRNA and vortexed. The reaction was shaken for 1 hr at RT, followed by ethanol precipitation.

Synthesis of MC1011: To 0.1 M sodium phosphate buffer pH 7.4 (12.5 μL) was added MC1010 (25 μg, 12.5 μL of 2 μg/μL H₂O, 29 nmol) and vortexed. mPegSPA 5k (140 μg, 2.8 μL of 50 μg/μL DMSO, 29 nmol, Shearwater Chemical) was added to the siRNA and vortexed. The reaction was shaken for 1 hr at RT, followed by ethanol precipitation.

Synthesis of MC1012: To 0.1M sodium phosphate buffer pH 7.4 (12.5 μL) was added MC1010 (25 μg, 12.5 μL of 2 μg/μL H₂O, 29 nmol) and vortexed. mPeg₂NHS 10k (290 μg, 5.8 μL of 50 μg/μL DMSO, 29 nmol, Shearwater Chemical) was added to the siRNA and vortexed. The reaction was shaken for 1 hr at RT, followed by ethanol precipitation.

Synthesis of MC1013: To 0.1M sodium phosphate buffer pH 7.4 (12.5 μL) was added MC1010 (25 μg, 12.5 mL of 2 μg/μL H₂O, 29 nmol) and vortexed. mPeg₂NHS 20k (570 μg, 11 μL of 50 μg/μL DMSO, 29 nmol, Shearwater Chemical) was added to the siRNA and vortexed. The reaction was shaken for 1 hr at RT, followed by ethanol precipitation.

Each modified siRNA was brought up in 25 μL H₂O (2 μg/μL) and stored at −20° C.

### Example 7

### In Vitro siRNA Induced Knockdown in CHO-L UC Cells

Samples were formulated as follows:

Sample 1: 150 mM NaCl (100 μL)

Sample 2: 150 mM NaCl (100 μL)+GL3 (1 μL, 100 ng, 0.0075 pmol)

Sample 5: 150 mM NaCl (100 μL)+GL3 (1 μL, 100 ng, 0.0075 pmol)+TransIT-TKO (TKO)

Sample 6, 10, 14, 18, 22: 150 mM NaCl (100 μL)+modified GL3 (1 μL, 100 ng, 0.0075 pmol)

Sample 9, 13, 17, 21, 25: 150 mM NaCl (100 μL)+modified GL3 (1 μL, 100 ng, 0.0075 pmol)+TransIT-TKO

Transfection of CHO-Luc Cells. Samples were prepared as above. Transfections were conducted in duplicate in 12 well plates by covering the cells with 500 μL DMEM with 10% serum and adding 100 μL of transfection sample. Cells were harvested 24 hr post transfection, and read on a luminometer. RLUs are the average of the two wells.

| | siRNA | | Mean | | |
|---|---|---|---|---|---|
| # | (12.5 nM) | TKO (μl) | RLU | % Expression | % Confluency |
| 1 | none | — | 1,188,440 | 100 | 95 |
| 2 | GL3 | — | 1,001,832 | 84 | 88 |
| 5 | GL3 | 3 | 168,116 | 14 | 75 |
| 6 | MC999 | — | 1,081,745 | 91 | 85 |
| 9 | MC999 | 3 | 529,155 | 45 | 93 |
| 10 | MC1003 | — | 975,781 | 82 | 95 |
| 13 | MC1003 | 3 | 427,072 | 36 | 95 |
| 14 | MC1000 | — | 1,015,834 | 85 | 93 |
| 17 | MC1000 | 3 | 386,305 | 33 | 75 |
| 18 | MC1004 | — | 952,107 | 80 | 95 |
| 21 | MC1004 | 3 | 733,820 | 62 | 95 |
| 22 | MC1001 | — | 947,359 | 80 | 95 |
| 25 | MC1001 | 3 | 637,221 | 54 | 88 |

The results show that the modifications do not inactivate the siRNAs or impair their ability to be delivered to cells by the TransIT-TKO transfection reagent. Also, the use of modified siRNA does not cause cellular toxicity.

### Example 8

### In Vivo Delivery and Gene Expression Knockdown Using Modified siRNA

Several complexes were prepared as follows:

Complex I. To 9.8 mL Ringers was added 40 pg pGL3-control and 4 gg pRLSV40. To 200 μL 150 mM NaCl was added 20 ug MC999. The 150 mM NaCl solution was added to the Ringers solution and vortexed.

Complex II. To 9.8 mL Ringers was added 40 pg pGL3-control and 4 μg pRLSV40. To 200 μL 150 mM NaCl was added 20 μg GL3. The 150 mM NaCl solution was added to the Ringers solution and vortexed.

Complex III. To 9.8 mL Ringers was added 40 μg pGL3-control and 4 μg pRLSV40. To 200 μL 150 mM NaCl was added 20 μg EGFP. The 150 mM NaCl solution was added to the Ringers solution and vortexed.

Complex IV. To 9.8 mL Ringers was added 40 μg pGL3-control and 4 μg pRLSV40. To 200 μL 150 mM NaCl was added 20 μg MC999. The 150 mM NaCl solution was added to the Ringers solution and vortexed.

Complex V. To 9.8 mL Ringers was added 40 μg pGL3-control and 4 μg pRLSV40. To 200 μL 150 mM NaCl was added 20 μg MC1007. The 150 mM NaCl solution was added to the Ringers solution and vortexed.

Complex VI. To 9.8 mL Ringers was added 40 μg pGL3-control and 4 μg pRLSV40. To 200 μL 150 mM NaCl was added 20 μg MC1001. The 150 mM NaCl solution was added to the Ringers solution and vortexed.

Complex VII. To 9.8 mL Ringers was added 40 μg pGL3-control and 4 μg pRLSV40. To 200 μL 150 mM NaCl was added 20 μg MC1009. The 150 mM NaCl solution was added to the Ringers solution and vortexed.

Complex VIII. To 9.8 mL Ringers was added 40 μg pGL3-control and 4 μg pRLSV40. To 200 μL 150 mM NaCl was added 20 μg MC1003. The 150 mM NaCl solution was added to the Ringers solution and vortexed.

Complex IX. To 9.8 mL Ringers was added 40 μg pGL3-control and 4 μg pRLSV40. To 200 μL 150 mM NaCl was added 20 μg MC1011. The 150 mM NaCl solution was added to the Ringers solution and vortexed.

Complex X. To 9.8 mL Ringers was added 40 μg pGL3-control and 4 μg pRLSV40. To 200 μL 150 mM NaCl was added 20 μg MC1005. The 150 mM NaCl solution was added to the Ringers solution and vortexed.

Complex XI. To 9.8 mL Ringers was added 40 μg pGL3-control and 4 μg pRLSV40. To 1 To 200 μL 150 mM NaCl was added 20 μg MC1013. The 150 mM NaCl solution was added to the Ringers solution and vortexed.

2.5 mL tail vein injections of 2.5 mL of the complex were preformed on ICR mice (n=3) using a 30 gauge, 0.5 inch needle. One day after injection, the animal was sacrificed, and a dual luciferase assay was conducted on the liver. Luciferase and Renilla expression was determined on a Centro LB960 plate luminometer (Berthold Technologies).

| siRNA | | | LUC/ |
|---|---|---|---|
| (see example 6) | LUC | Renilla | Renilla* |
| | 101,699,293 | 120,568,450 | 90.3% |
| GL3 | 16,403,927 | 217,429,930 | 7.5% |
| EGFP | 120,625,927 | 154,883,747 | 86.1% |
| MC999 (GL3) | 77,096,170 | 128,761,200 | 62.0% |
| MC1007 (EGFP) | 78,703,243 | 75,986,633 | 107.2% |
| MC1001 (GL3) | 83,151,933 | 215,030,733 | 40.4% |
| MC1009 (EGFP) | 95,901,940 | 92,716,783 | 105.6% |
| MC1003 (GL3) | 98,824,690 | 194,249,110 | 51.0% |
| MC1011 (EGFP) | 189,568,540 | 255,097,493 | 74.9% |
| MC1005 (GL3) | 76,387,267 | 90,927,400 | 86.7% |
| MC1013 (EGFP) | 114,399,427 | 141,435,220 | 80.3% |

*average of ratios determined for individual mice (n = 3)

### Example 9

### Alkylation of siRNAs to form an Amine-Modified siRNAs, and Their Reaction with Peg Derivatives

Synthesis of GL3-NH₂ (2 eq). To H₂O (103 μL) was added GL3 (250 μg, 147 μL of 1.7 μg/μL, 19 nmol, Dharmacon) and gently mixed. Label-It Amine (10 μg, 1.0 μL of 10 μg/μL DMSO, 37 nmol) was added and vortexed followed by the addition of 1N NaOH (1 μL). The reaction was incubated at 37° C. for 1 hr. The reaction was removed from heat. After the reaction reached ambient temperature, the modified siRNA was ethanol precipitated. The pellet was brought up in H₂O (50 μL, 5 μg/μL) and stored at −20° C.

Synthesis of GL3-Peg5k(2): GL3-NH₂ (2 eq) with mPegSPA 5k. To 0.1M sodium phosphate buffer pH 7.4 (39.3 μL) was added GL3-NH₂ (2 eq) (50 μg, 10 μL of 5 μg/μL H₂O, 34 nmol) and vortexed. mPegSPA 5k (0.37 μg, 7 μL of 50 μg/μL DMSO, 7.4 nmol, Shearwater Chemical) was added to the siRNA and vortexed. The reaction was shaken for 1 hr at RT. Final concentration was 1 μg/μL.

Synthesis of GL3-Peg10k(2): GL3-NH₂ (2 eq) with mPeg₂NHS 10k. To 0.1M sodium phosphate buffer pH 7.4 (38.5 μL) was added GL3-NH₂ (2 eq) (50 μg, 10 μL of 5 μg/μL H₂O, 34 nmol) and vortexed. mPeg₂NHS 10k (74 μg, 1.5 μL of 50 μg/μL DMSO, 7.4 nmol, Shearwater Chemical) was added to the siRNA and vortexed. The reaction was shaken for 1 hr at RT. Final concentration was 1 μg/μL.

Synthesis of GL3-Peg20k(2): GL3-NH₂ (2 eq) with mPeg₂NHS 20k. To 0.1 M sodium phosphate buffer pH 7.4 (37 μL) was added GL3-NH₂ (2 eq) (50 μg, 10 μL of 5 μg/μL H₂O, 34 nmol) and vortexed. mPeg₂NHS 10k (150 μg, 3 μL of 50 μg/μL DMSO, 7.4 nmol, Shearwater Chemical) was added to the siRNA and vortexed. The reaction was shaken for 1 hr at RT. Final concentration was 1 μg/μL.

Synthesis of GL3-NH₂ (5 eq). To H₂O (103 μL) was added GL3 (250 μg, 147 μL of 1.7 μg/μL, 19 nmol, Dharmacon) and gently mixed. Label-It Amine (25 μg, 2.5 μL of 10 μg/μL DMSO, 93 nmol) was added and vortexed followed by the addition of 1N NaOH (1 μL). The reaction was incubated at 37° C. for 1 hr. The reaction was removed from heat. After the reaction reached ambient temperature, the modified siRNA was ethanol precipitated. The pellet was brought up in H₂O (50 μL, 5 μg/μL) and stored at −20° C.

Synthesis of GL3-Peg5k(5): GL3-NH₂ (5 eq) with mPegSPA 5k. To 0.1 M sodium phosphate buffer pH 7.4 (38.1 μL) was added GL3-NH₂ (5 eq) (50 μg, 10 μL of 5 μg/μL H₂O, 34 nmol) and vortexed. mPegSPA 5k (193 μg, 9 μL of 50 μg/μL DMSO, 19 nmol, Shearwater Chemical) was added to the siRNA and vortexed. The reaction was shaken for 1 hr at RT. Final concentration was 1 μg/μL.

Synthesis of GL3-Peg10k(5): GL3-NH₂ (5 eq) with mPeg₂NHS 10k. To 0.1M sodium phosphate buffer pH 7.4 (36.2 μL) was added GL3-NH₂ (5 eq) (50 μg, 10 μL of 5 μg/μL H₂O, 34 nmol) and vortexed. mPeg₂NHS 10k (190 μg, 3.8 μL of 50 μg/μL DMSO, 19 nmol, Shearwater Chemical) was added to the siRNA and vortexed. The reaction was shaken for 1 hr at RT. Final concentration was 1 μg/μL.

Synthesis of GL3-Peg20k(5): GL3-NH₂ (5 eq) with mPeg₂NHS 20k. To 0.1M sodium phosphate buffer pH 7.4 (32.6 μL) was added GL3-NH₂ (5 eq) (50 μg, 10 μL of 5 μg/μL H₂O, 34 nmol) and vortexed. mPeg₂NHS 20k (370 μg, 7.4 μL of 50 μg/μL DMSO, 19 nmol, Shearwater Chemical) was added to the siRNA and vortexed. The reaction was shaken for 1 hr at RT. Final concentration was 1 μg/μL.

Synthesis of EGFP-NH₂ (2 eq). To H₂O (231.4 μL) was added EGFP (250 μg, 18.6 μL of 13.4 μg/μL, 19 nmol, Dharmnacon) and gently mixed. Label-It Amine (10 μg, 1.0 μL of 10 μg/μL DMSO, 37 nmol) was added and vortexed followed by the addition of 1N NaOH (1 μL). The reaction was incubated at 37° C. for 1 hr. The reaction was removed from heat. After the reaction reached ambient temperature, the modified siRNA was ethanol precipitated. The pellet was brought up in H₂O (50 μL, 5 μg/μL) and stored at −20° C.

Synthesis of EGFP-Peg5k(2): EGFP-NH₂ (2 eq) with mPegSPA 5k. To 0.1 M sodium phosphate buffer pH 7.4 (39.2 μL) was added EGFP-NH₂ (2 eq) (50 μg, 10 μL of 5 μg/μL H₂O, 45 nmol) and vortexed. mPegSPA 5k (41 μg, 0.82 μL of 50 μg/μL DMSO, 8.4 nmol, Shearwater Chemical) was added to the siRNA and vortexed. The reaction was shaken for 1 hr at RT. Final concentration was 1 μg/μL.

Synthesis of EGFP-Peg10k(2): EGFP-NH₂ (2 eq) with mPeg₂NHS 10k. To 0.1 M sodium phosphate buffer pH 7.4 (38.3 μL) was added EGFP-NH₂ (2 eq) (50 μg, 10 μL of 5 μg/μL H₂O, 45 nmol) and vortexed. mPeg₂NHS 10k (84 μg, 1.7 μL of 50 μg/μL DMSO, 8.4 nmol, Shearwater Chemical) was added to the siRNA and vortexed. The reaction was shaken for 1 hr at RT. Final concentration was 1 μg/μL.

Synthesis of EGFP-Peg20k(2): EGFP-NH₂ (2 eq) with mPeg₂NHS 20k. To 0.1 M sodium phosphate buffer pH 7.4 (36.6 μL) was added EGFP-NH₂ (2 eq) (50 μg, 10 μL of 5 μg/μL H₂₀, 45 nmol) and vortexed. mPeg₂NHS 10k (170 μg, 3.4 μL of 50 μg/μL DMSO, 8.4 nmol, Shearwater Chemical) was added to the siRNA and vortexed. The reaction was shaken for 1 hr at RT. Final concentration was 1 μg/μL.

Synthesis of EGFP-NH₂ (5 eq). To H₂O (231.4 μL) was added EGFP (250 μg, 18.6 μL of 13.4 μg/μL, 19 nmol, Dharmacon) and gently mixed. Label-It Amine (25 μg, 2.5 μL of 10 μg/μL DMSO, 93 nmol) was added and vortexed followed by the addition of 1N NaOH (1 μL). The reaction was incubated at 37° C. for 1 hr. The reaction was removed from heat. After the reaction reached ambient temperature, the modified siRNA was ethanol precipitated. The pellet was brought up in H₂O (50 μL, 5 μg/μL) and stored at −20° C.

Synthesis of EGFP-Peg5k(5): EGFP-NH₂ (5 eq) with mPegSPA 5k. To 0.1M sodium phosphate buffer pH 7.4 (38.3 μL) was added EGFP-NH₂ (5 eq) (50 μg, 10 μL of 5 μg/μL H₂O, 45 nmol) and vortexed. mPegSPA 5k (84 μg, 1.7 μL of 50 μg/μL DMSO, 17 nmol, Shearwater Chemical) was added to the siRNA and vortexed. The reaction was shaken for 1 hr at RT. Final concentration was 1 μg/μL.

Synthesis of EGFP-Peg 10k(5): EGFP-NH₂ (5 eq) with mPeg₂NHS 10k. To 0.1 M sodium phosphate buffer pH 7.4 (36.6 μL) was added EGFP-NH₂ (5 eq) (50 μg, 10 μL of 5 μg/μL H₂O, 45 mmol) and vortexed. mPeg₂NHS 10k (170 μg, 17 3.4 μL of 50 μg/μL DMSO, nmol, Shearwater Chemical) was added to the siRNA and vortexed. The reaction was shaken for 1 hr at RT. Final concentration was 1 μg/μL.

Synthesis of EGFP-Peg20k(5): EGFP-NH₂ (5 eq) with mPeg₂NHS 20k. To 0.1 M sodium phosphate buffer pH 7.4 (33.2 μL) was added EGFP-NH₂ (5 eq) (50 μg, 10 μL of 5 μg/μL H₂O, 45 nmol) and vortexed. mPeg₂NHS 20k (340 μg, 6.8 μL of 50 μg/μL DMSO, 17 nmol, Shearwater Chemical) was added to the siRNA and vortexed. The reaction was shaken for 1 hr at RT. Final concentration was 1 μg/μL.

### Example 10

### In Vivo Delivery and Gene Expression Knockdown Using Modified siRNA

Several complexes were prepared as follows:

Complex I. To Ringers (9.8 mL) was added pGL3-control (a plasmid with a SV40 promoter driving the Luciferase expression cassette, 40 μg, 20 μL of 2 μg/μL solution in water) followed by pRLSV40 (a plasmid with a SV40 promoter driving the Renilla Luciferase expression cassette 4 μg, 2 μL of 2 μg/μL). To 150 mM NaCl (200 μL) was added MC999 (20 μg, 1.5 μL) and vortexed. The 150 mM NaCl solution was added to the Ringers solution and vortexed.

Complex II. To Ringers (9.8 mL) was added pGL3-control (40 μg, 20 μL of 2 μg/μL solution in water) followed by pRLSV40 (4 μg, 2 μL of 2 μg/μL). To 150 mM NaCl (200 μL) was added GL3 (20 μg, 1.3 μL of 13.3 μg/μL water solution) and vortexed. The 150 mM NaCl solution was added to the Ringers solution and vortexed.

Complex III. To Ringers (9.8 mL) was added pGL3-control (40 μg, 20 μL of 2 μg/μL solution in water) followed by pRLSV40 (4 μg, 2 μL of 2 μg/μL). To 150 mM NaCl (200 μL) was added EGFP (20 μg, 1.5 μL of 13.3 μg/μL water solution) and vortexed. The 150 mM NaCl solution was added to the Ringers solution and vortexed.

Complex IV. To Ringers (9.8 mL) was added pGL3-control (40 μg, 20 μL of 2 μg/μL solution in water) followed by pRLSV40 (4 μg, 2 mL of 2 pg/pL). To 150 mM NaCl (200 μL) was added GL3-Peg5k(2) (20 μg, 20 μL of 1 μg/μL water solution) and vortexed. The 150 mM NaCl solution was added to the Ringers solution and vortexed.

Complex V. To Ringers (9.8 mL) was added pGL3-control (40 μg, 20 μL of 2 μg/μL solution in water) followed by pRLSV40 (4 μg, 2 μL of 2 μg/μL). To 150 mM NaCl (200 μL) was added EGFP-Peg5k(2) (20 μg, 20 μL of 1 μg/μL water solution) and vortexed. The 150 mM NaCl solution was added to the Ringers solution and vortexed.

Complex VI. To Ringers (9.8 mL) was added pGL3-control (40 μg, 20 μL of 2 μg/μL solution in water) followed by pRLSV40 (4 μg, 2 μL of 2 μg/μL). To 150 mM NaCl (200 μL) was added GL3-Peg5k(5) (20 μg, 20 μL of 11 g/μL water solution) and vortexed. The 150 mM NaCl solution was added to the Ringers solution and vortexed.

Complex VII. To Ringers (9.8 mL) was added pGL3-control (40 μg, 20 μL of 2 μg/μL solution in water) followed by pRLSV40 (4 μg, 2 μL of 2 μg/μL). To 150 mM NaCl (200 μL) was added EGFP-Peg5k(5) (20 μg, 20 μL of 1 μg/μL water solution) and vortexed. The 150 mM NaCl solution was added to the Ringers solution and vortexed.

Complex VIII. To Ringers (9.8 mL) was added pGL3-control (40 μg, 20 μL of 2 μg/μL solution in water) followed by pRLSV40 (4 μg, 2 μL of 2 μg/μL). To 150 mM NaCl (200 μL) was added GL3-Peg20k(2) (20 μg, 20 μL of 1 μg/μL water solution) and vortexed. The 150 mM NaCl solution was added to the Ringer's solution and vortexed.

Complex IX. To Ringers (9.8 mL) was added pGL3-control (40 μg, 20 μL of 2 μg/μL solution in water) followed by pRLSV40 (4 μg, 2 μL of 2 μg/μL). To 150 mM NaCl (200 μL) was added EGFP-Peg20k(2) (20 μg, 20 μL of 1 μg/μL water solution) and vortexed. The 150 mM NaCl solution was added to the Ringers solution and vortexed.

Complex X. To Ringers (9.8 mL) was added pGL3-control (40 μg, 20 mL of 2 μg/μL solution in water) followed by pRLSV40 (4 μg, 2 μL of 2 μg/μL). To 150 mM NaCl (200 μL) was added GL3-Peg20k(5) (20 μg, 20 μL of 1 μg/μL water solution) and vortexed. The 150 mM NaCl solution was added to the Ringers solution and vortexed.

Complex XI. To Ringers (9.8 mL) was added pGL3-control (40 μg, 20 μL of 2 μg/μL solution in water) followed by pRLSV40 (4 μg, 2 μL of 2 μg/μL). To 150 mM NaCl (200 μL) was added EGFP-Peg20k(5) (20 μg, 20 μL of 1 μg/μL water solution) and vortexed. The 150 mM NaCl solution was added to the Ringers solution and vortexed.

2.5 mL tail vein injections of 2.5 mL of the complex were preformed on ICR mice (n=3) using a 30 gauge, 0.5 inch needle. One day after injection, the animal was sacrificed, and a dual luciferase assay was conducted on the liver. Luciferase and Renilla expression was determined on a Centro LB960 plate luminometer (Berthold Technologies).

Results: Dual Luciferase Assay of Livers. All cells were transfected with the pGL3-control and pRLSV40 luciferase expression plasmids.

| | siRNA | | | |
|---|---|---|---|---|
| siRNA | modification | LUC | REN | Luc/Ren |
| | — | 88,909,490 | 76,545,837 | 120 |
| GL3 | — | 13,364,907 | 94,569,847 | 15 |
| EGFP control | — | 64,526,160 | 50,677,823 | 122 |
| GL3 | Peg5k(2) | 116,073,260 | 140,923,633 | 82 |
| EGFP control | Peg5k(2) | 122,944,920 | 112,250,317 | 110 |
| GL3 | Peg5k(5) | 16,075,750 | 106,257,890 | 15 |
| EGFP control | Peg5k(5) | 67,554,737 | 89,084,540 | 69 |
| GL3 | Peg20k(2) | 109,951,353 | 112,490,970 | 105 |
| EGFP control | Peg20k(2) | 122,728,397 | 132,728,220 | 102 |
| GL3 | Peg20k(5) | 18,632,693 | 90,463,347 | 20 |
| EGFP control | Peg20k(5) | 79,473,323 | 73,580,060 | 112 |

The results that the GL3-Peg(5) modified siRNAs are fully active when delivered to cells in vivo.

### Example 11

### In Vitro Delivery of Modified siRNA to CHO-Luc Cells and Knockdown of Luciferase Expression

Samples were formulated as follows:

Sample 1. OPTI (100 μL)

Sample 2. OPTI (100 μL)+GL3 siRNA (100 ng, 1 μL of 100 ng/μL water solution, 0.0075 pmol, Dharmacon)+TransIT-TKO transfection agent (2 μL of 2 μg/μL EtOH).

Sample 3, 6, 9-12. OPTI (100 μL)+modified GL3 siRNA (1 μL of 100 ng/μL water solution, 100 ng, 0.0075 pmol).

Sample 4-5,7-8, 13-24. OPTI (100 μL)+modified GL3 siRNA (100 ng, 1 μL of 100 ng/μL water solution, 0.0075 pmol)+TransIT-TKO transfection agent (2 μg/μL EtOH).

Transfection of CHO-Luc Cells. Samples were prepared as above. Transfections were conducted in duplicate in 12 well plates by covering the cells with 500 μL DMEM with 10% serum and adding 100 μL of transfection sample. Cells were harvested 24 hr post transfection, and read on a luminometer. RLUs are the average of the two wells.

| | | TKO | | % |
|---|---|---|---|---|
| # | Sample | (μl) | Mean RLU | Expression |
| 1 | OPTI | — | 2,405,369 | 100 |
| 2 | GL3 | 2 | 506,868 | 21 |
| 3 | GL3-NH2(2) | — | 2,068,001 | 86 |
| 4 | GL3-NH2(2) | 2 | 2,450,345 | 102 |
| 5 | GL3-NH2(2) | 3 | 1,819,653 | 76 |
| 6 | GL3-NH2(5) | — | 1,943,917 | 81 |
| 7 | GL3-NH2(5) | 2 | 1,091,177 | 45 |
| 8 | GL3-NH2(5) | 3 | 463,029 | 19 |
| 9 | GL3-Peg5k(2) | — | 2,226,878 | 93 |
| 14 | GL3-Peg5k(2) | 2 | 2,391,703 | 99 |
| 15 | GL3-Peg5k(2) | 3 | 1,746,008 | 73 |
| 10 | GL3-Peg5k(5) | — | 2,330,134 | 97 |
| 17 | GL3-Peg5k(5) | 2 | 1,061,823 | 44 |
| 18 | GL3-Peg5k(5) | 3 | 598,835 | 25 |
| 11 | GL3-Peg20k(2) | — | 2,215,972 | 92 |
| 20 | GL3-Peg20k(2) | 2 | 2,215,888 | 92 |
| 21 | GL3-Peg20k(2) | 3 | 1,848,630 | 77 |
| 12 | GL3-Peg20k(5) | — | 1,965,598 | 82 |
| 23 | GL3-Peg20k(5) | 2 | 978,274 | 41 |
| 24 | GL3-Peg20k(5) | 3 | 565,093 | 23 |

Unmodified GL3 siRNA and GL3-NH2(5)±PEG siRNA were delivered to CHO cells with the TransIT-TKO transfection agent and efficiently knocked down expression of the luciferase gene.

### Example 12

### In Vitro Delivery of Modified siRNA to HEPA-Luc Cells and Knockdown of Luciferase Expression. Samples Were Formulated as Follows:

Sample 1. OPTI (100 μL)

Sample 2. OPTI (100 μL)+GL3 siRNA (100 ng, 1 μL of 100 ng/μL water solution).

Sample 3. OPTI (100 μL)+TransIT-TKO (6 μL of 2 μg/μL EtOH).

Sample 4-6. OPTI (100 μL)+GL3 siRNA (100 ng, 1 μL of 100 ng/μL water solution)+TransIT-TKO (2 μg/μL EtOH).

Sample 7, 11, 15, 19, 23, 27. OPTI (100 μL)+modified GL3 (100 ng, 1 μL of 100 ng/μL water solution).

Sample 8-10, 12-14, 16-18, 20-22, 24-26, 28-30. To OPTI (100 μL)+modified GL3 (100 ng, 1 μL of 100 ng/μL water solution)+TransIT-TKO (2 μg/μL EtOH).

Transfection of Hepa-Luc Cells. Samples were prepared as above. Transfections were conducted in duplicate in 12 well plates by covering the cells with 500 μL DMEM with 10% serum and adding 100 μL of transfection sample. Cells were harvested 48 hr post transfection, and read on a luminometer. RLUs are the average of the two wells.

| | | TKO | Mean | | |
|---|---|---|---|---|---|
| # | Sample | (μl) | RLU | % Expression | % Confluency |
| 1 | OPT1 | — | 9,104,387 | 100 | 100 |
| 2 | GL3 | — | 8,353,833 | 92 | 100 |
| 3 | TKO (6 μL) | — | 7,121,195 | 78 | 93 |
| 4 | GL3 | 4 | 4,515,103 | 50 | 98 |
| 5 | GL3 | 5 | 3,067,717 | 34 | 93 |
| 6 | GL3 | 6 | 2,216,579 | 24 | 93 |
| 11 | GL3-NH2(5) | — | 8,583,318 | 94 | 98 |
| 12 | GL3-NH2(5) | 4 | 5,731,910 | 63 | 98 |
| 13 | GL3-NH2(5) | 5 | 4,851,552 | 53 | 98 |
| 14 | GL3-NH2(5) | 6 | 4,865,578 | 53 | 93 |
| 19 | GL3-Peg5k(5) | — | 8,128,660 | 89 | 100 |
| 20 | GL3-Peg5k(5) | 4 | 6,386,829 | 70 | 100 |
| 21 | GL3-Peg5k(5) | 5 | 4,661,432 | 51 | 93 |
| 22 | GL3-Peg5k(5) | 6 | 3,353,224 | 37 | 93 |
| 27 | GL3-Peg20k(5) | — | 8,400,584 | 92 | 100 |
| 28 | GL3-Peg20k(5) | 4 | 6,483,604 | 71 | 100 |
| 29 | GL3-Peg20k(5) | 5 | 4,438,972 | 49 | 90 |
| 30 | GL3-Peg20k(5) | 6 | 3,973,030 | 44 | 88 |

Unmodified GL3 siRNA and GL3-NH2(5)+PEG siRNA were delivered to Hepa cells with the TransIT-TKO transfection agent and efficiently knocked down expression of the luciferase gene.

### Example 13

Post-synthetic amine-modification ofsiRNA increases nuclease protection. One reason for modification of the siRNA is to protect the siRNA from degradation by nucleases. A method used in the art to protect nucleic acids from nuclease digestion is to synthesis the nucleic acid with a nonstandard ribose backbone, such as in a phosphorothioate oligonucleotide. We show here, that modification of hydroxyls in the backbone of a phosphodiester siRNA, protects the siRNA from RNAse I digection. RNase I is a known enzyme that cleaves RNA at phosphodiester bonds between nucleotides. Samples were prepared as follows:

Sample 1. H₂O (5.5 μL)+GL3 siRNA (250 ng, 2.5 μL of 100 ng/μL, Dharmacon).

Sample 2. H₂O (0.5 μL)+GL3 siRNA (250 ng, 2.5 μL of 100 ng/μL)+RNase I (25 U, 5 μL of 5 units/μL).

Sample 3. (5.5 μL)+GL3-NH₂(2) siRNA (250 ng, 2.5 μL of 100 ng/μL).

Sample 4. H₂O (0.5 μL)+GL3-NH₂(2) siRNA (250 ng, 2.5 μL of 100 ng/μL)+RNase I (25 U, 5 μL of 5 U/μL).

All samples were incubated at RT for 30 min. Loading buffer (2 μL of 3×, Invitrogen) was added to each sample and mixed. Samples were loaded into a 20% TB gel and run at 180 V in 1× TBE buffer for 30 min. The gel was stained with EtBr (0.5 μg/mL in 1× TAE buffer) and visualized on a UV light box.

| Lane | Sample | 50 units RNAse I |
|---|---|---|
| 1 | GL3 (250 ng) | − |
| 2 | GL3 (250 ng) | + |
| 3 | GL3-NH2(2) (250 ng) | − |
| 4 | GL3-NH2(2) (250 ng) | + |

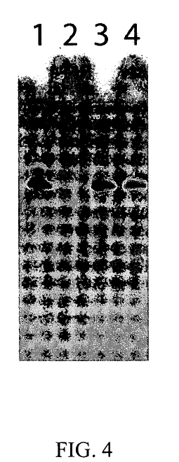

FIG. 4 shows an electrophoresis gel of amine-modified siRNA demonstrating that the modified siRNA is protected from nuclease degradation.

### Example 14

### Post-Synthetic Hydroxyl Modification of siRNA Increases Nuclease Protection

Samples were prepared as follows.

Sample 1. H₂O (7.5 μL)+DNA Ladder (0.5 μL, 1 kb Invitrogen).

Sample 2. H₂O (6.5 μL)+GL3 siRNA (2.5 μg, 1.5 μL of 1.7 μg/μL, Dharmacon).

Sample 3. H₂O (1.5 μL)+GL3 siRNA (2.5 μg, 1.5 μL of 1.7 μg/μL)+RNase I (50 u, 5 μL of 10 u/μL).

Sample 4. H₂O (6.75 μL)+GL3-Lauroyl-1 siRNA (2.5 μg, 1.25 μL of 2 μg/μL DMF).

Sample 5. H₂O (1.75 μL)+GL3-Lauroyl-1 siRNA (2.5 μg, 1.25 μL of 2 μg/μL DMF)+RNase I (50 u, 5 μL of 10 u/μL).

Sample 6. H₂O (6.75 μL)+GL3-Lauroyl-2 siRNA (2.5 μg, 1.25 μL of 2 μg/μL DMF).

Sample 7. H₂O (1.75 μL)+GL3-Lauroyl-2 siRNA (2.5 μg, 1.25 μL of 2 μg/μL DMF)+RNase 1 (50 u, 5 μL of 10 u/μL).

Sample 8. H₂O (6.75 μL)+GL3-Lauroyl-3 siRNA (2.5 μg, 1.25 μL of 2 μg/μL DMF).

Sample 9. H₂O (1.75 μL)+GL3-Lauroyl-3 siRNA (2.5 μg, 1.25 μL of 2 μg/μL DMF)+RNase I (50 u, 5 μL of 10 u/μL).

All samples were incubated at RT for 1 hr. Loading buffer (2 μL of 3×) was added to each sample and mixed. Samples were loaded into a 20% TB gel and run at 180 V in 1× TBE buffer for 30 min. The gel was stained with EtBr (0.5 μg/mL in 1× TAE buffer) and visualized on a UV light box.

| Lane | Sample | 50 units RNAse I |
|---|---|---|
| 1 | DNA Ladder (1 kb) | − |
| 2 | GL3 (2.5 μg) | − |
| 3 | GL3 (2.5 μg) | + |
| 4 | GL3-Lauroyl-1 (2.5 μg) | − |
| 5 | GL3-Lauroyl-1 (2.5 μg) | + |
| 6 | GL3-Lauroyl-2 (2.5 μg) | − |
| 7 | GL3-Lauroyl-2 (2.5 μg) | + |
| 8 | GL3-Lauroyl-3 (2.5 μg) | − |
| 9 | GL3-Lauroyl-3 (2.5 μg) | + |
| 10 | blank | |

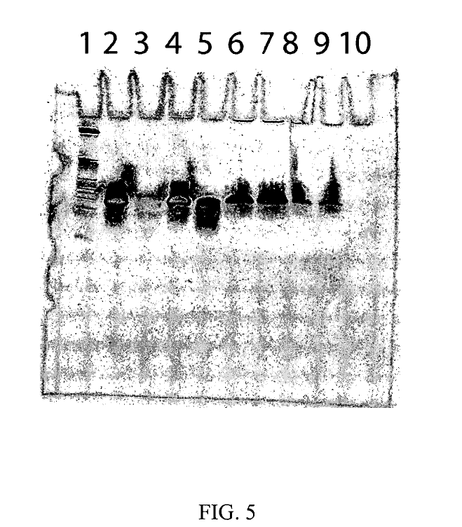

FIG. 5 shows an electrophoresis gel of hydroxyl modified siRNA demonstrating that the modified siRNA is protected from nucleiase degradation.

### Example 15

### In Vivo Delivery and Cellular Uptake of Modified siRNA's

Part A. Preparation of Cy3 labeled GL3 siRNA. To H₂O (425 μL) was added GL3 siRNA (1000 μg, 75 μL of 13.3 μg/μL solution, 75 nmol) and the solution was mixed with vortexing. Label-IT Cy3 (400 μg, 8 μL of 50 μg/μL in DMSO) was added and the resulting solution was mixed with vortexing. 1 N NaOH (2 μL) was added immediately to the solution while vortexing. The solution was incubated at 37° C. for 1 hr. The reaction was removed from heat and allowed to cool to ambient temperature, followed by ethanol precipitation. The pellet was dissolved in H₂O (500 μL, 2 μg/mL) and stored at −20° C.

Part B. Modification of Cy3-labeled GL3 siRNA, silylation of dsRNA with chloro-dimethyloctadecylsilane. To 200 μg of Cy3-GL3 siRNA (14.6 μL of a 13.7 μg/μL solution in water, 0.015 mmol dsRNA, 0.63 μmol —OH) was added 100 μL of anhydrous dimethylformamide and the solution was concentrated to dryness. The resulting solid was resuspended in 400 μL of anhydrous dimethylformamide. To the resulting solution was added chlorodimethyloctadecylsilane (2.6 mg, 0.0075 mmol), and diisopropylethylamine (1.3 μL, 0.0075 mmol). The solution was stirred for 4 hrs to afford Cy3-GL3-OSiC18.

Part C. Modification of Cy3 labeled GL3 siRNA, acylation with lauroylimidazole. To 200.0 μg of Cy3-GL3 siRNA (14.6 μL of a 13.7 ng/μL solution in water, 0.015 μmnol dsRNA, 0.63 μmol —OH) was added 185 μL anhydrous dimethylformamide. To this solution was added 140 μg (0.63 μmol) lauroyl chloride and 210 μg (3.2 μmol) imidazole in 200 μL DMF. The resulting solution was stirred at RT for 4 hr to afford Cy3-GL3-OLauroyl.

Part D. Delivery of Modified, Labeled siRNAs via Mouse Portal Vein. Several complexes were prepared for portal vein injection. MC1054 is a cholesterol modified cell targeting peptide (Chol-KNESSTNATNTKQWRDETKGFRDEARRFKNTAG-OH, SEQ ID 7). The N-terminus of the peptide is capped with cholesterol chloroformate. The crude peptide was purified by HPLC chromatography to a greater than 94% purity level. CholMel is a cholesterol modified membrane active peptide (Chol-GIGAILKVLATGLPTLISWIKN-KRKQ-OH, SEQ ID 8). The N-terminus of the peptide is capped with cholesterol chloroformate. The crude peptide was purified by HPLC chromatography to a greater than 94% purity level.

Complex I. Cy3-GL3 siRNA (40 μL, 0.5 μg/μL in DMF).

Complex II. Cy3-GL3-OSi(CH₃)₂C₁₈H₃₇ (40 μL, 0.5 μg/μL in DMF).

Complex III. Cy3-GL3-OLauroyl (40 μL, 0.5 μg/L in DMF)

Complex IV. Cy3-GL3-OLauroyl (40 μL, 0.5 μg/μL in DMF)+MC1054 (20 μL, 1 μg/μL in DMF)+CholMel (20 μL, 1 μg/μL in DMF). The solution was vortexed and let sit for 30 min prior to use.

Mouse portal vein injections of complexes were conducted via a dual pump injection procedure. We used 2 Harvard Pumps (PHD 2000) with Hamilton (100 μL) and Becton Dickinson (1 mL) syringes connected together through a colliding flow mixing chamber to mix the DMF solution containing the modified siRNA together with isotonic glucose as the injection carrier solution. Typically mixtures were 0.67 μL of siRNA in DMF solution with 6.7 μL of isotonic glucose per second, with a total delivery volume of 220 μL (10 μg RNA) over 30 seconds for in vitro delivery. Livers were exposed through a ventral midline incision, and the complexes were injected over 30 sec into the portal vein using a 30-gauge, ½-inch needle. A microvessel clip was applied on the portal vein and the hepatic artery during the injection. Anesthesia was obtained from inhalation of isoflurane as needed. After 5 min, the animals were sacrificed and the livers harvested, sectioned, and examined under confocal laser scanning microscopy. Complex I showed no regions of cellular uptake or binding Cy3-GL3 siRNA. For complex II, some regions of the liver showed Cy3-RNA-OSiC 18 within hepatocytes, estimated at <5% of hepatocytes. For complex III, some regions of the liver showed Cy3-RNA-OLauroyl within hepatocytes, estimated at <5% of hepatocytes. A representative liver field is shown in FIG. 5 for complex IV. Several of the regions in the liver indicated strong hepatocyte uptake with the Cy3-GL3-OLauroyl/MC 1054/CholMel sample. Cy3-GL3-OLauroyl was observed in >10% of hepatocytes with some regions showing greater than 50% of hepatocytes. Additionally, Cy3-GL3-OLauroyl was observed within the nucleus of the hepatocytes at the 5 min harvest timepoint.

The foregoing is considered as illustrative only of the principles of the invention. Furthermore, since numerous modifications and changes will readily occur to those skilled in the art, it is not desired to limit the invention to the exact construction and operation shown and described. Therefore, all suitable modifications and equivalents fall within the scope of the invention.

We claim:

1. A composition for delivering an RNA to a mammalian cell comprising: a post-synthetically modified RNA.

2. The composition of claim 1 wherein the modified RNA consists of a functional group attached to the RNA.

3. The composition of claim 2 wherein the functional group is linked to the RNA via a labile bond.

4. The composition of claim 2 wherein the functional group is linked to a ribose 2′ hydroxyl of the RNA.

5. The composition of claim 3 wherein the functional group is selected from the list consisting of: hydrophobic group, membrane active compound, cell penetrating compound, cell targeting signal, interaction modifier, and steric stabilizer.

6. The composition of claim 4 wherein the modified RNA is modified at: a single ribose 2′ hydroxyl of the RNA, more than one but not all of the ribose 2′ hydroxyls of the RNA, or all of the ribose 2′ hydroxyls of the RNA.

7. The composition of claim 1 wherein the modified RNA consists of a silylated RNA.

8. The composition of claim 1 wherein the modified RNA consists of an acylated RNA.

9. The composition of claim 1 wherein the modified RNA consists of an alkylated RNA.

10. The composition of claim 2 wherein the composition further comprises a transfection agent.

11. The composition of claim 1 wherein the RNA is selected from the list consisting of: siRNA and microRNA.

12. The composition of claim 1 wherein the mammalian cell consists of: an in vivo mammalian cell or an in vitro mammalian cell.

13. The composition of claim 1 wherein the modified RNA is more resistant to nucleases than the same RNA if it were not modified.

14. A process for delivering an RNA to a mammalian cell comprising: post-synthetically modifying the RNA through silylation, acylation or alkylation to form a modified RNA, and contacting the cell with the modified RNA.

15. The process of claim 14 wherein modifying the RNA consists of covalently linking a functional group to a ribose 2′ hydroxyl of the RNA.

16. The process of claim 15 wherein the functional group is selected from the list consisting of: hydrophobic group, membrane active compound, cell penetrating compound, cell targeting signal, interaction modifier, and steric stabilizer.

17. The process of claim 14 wherein the modified RNA is complexed with a transfection agent.

18. The process of claim 17 wherein modifying the RNA increases interaction of the RNA with the transfection agent.

19. The process of claim 14 wherein modifying the RNA increases resistance of the RNA to degradation by nucleases.

20. The process of claim 14 wherein the RNA is selected from the list consisting of: siRNA and microRNA.
